(19) United States
(12) Patent Application Publication
(10) Pub. No.: US 2004/0167139 A1
(43) Pub. Date: Aug. 26, 2004

(54) Methods of treating cancer

(76) Inventor: David A. Potter, Indianapolis, IN (US)

Correspondence Address: FISH & RICHARDSON PC, 225 FRANKLIN ST, BOSTON, MA, 02110, US

(21) Appl. No.: 10/629,045

(22) Filed: Jul. 28, 2003

Related U.S. Application Data
(60) Provisional application No. 60/399,573, filed on Jul. 26, 2002.

(51) Int. Cl. A61K 31/513; A61K 31/427; A61K 31/34

(52) U.S. Cl. 514/269; 514/365; 514/473

(57) ABSTRACT

Methods for treating cancer are described herein. The methods include administering to an HIV-negative patient an m-calpain inhibitor such as ritonavir. Ritonavir or other m-calpain inhibitors can also be co-administered with other therapeutic agents such as a Cox-2 inhibitor, a taxane, or a proteasome inhibitor. Methods for determining whether a patient will respond to a particular method of treatment are also described herein.

### CROSS-REFERENCE TO RELATED APPLICATIONS

This application claims priority from U.S. Provisional Patent Application Serial No. 60/399,573, filed on Jul. 26, 2002, which is incorporated herein by reference in its entirety.

### TECHNICAL FIELD

This invention relates to the use of compounds such as ritonavir, lopinavir, amprenavir, indinavir and saquinavir in the treatment of cancer, including cancers associated with elevated levels of epidermal growth factor (EGF) receptor expression.

### BACKGROUND

Cancer is a leading cause of death in developed countries. Despite continuing advances in both diagnosis and treatment regimens, most existing treatments have undesirable side effects and limited efficacy. Progress in this field has been hindered because a number of different cellular events contribute to the formation and metastasis of tumors, and many of these events are still not well understood. Chemotherapy is one of the major options available for the first-line treatment in cancers, such as leukemia, and for second-line treatments of refractory solid tumors. Just as the cellular mechanisms that contribute to cellular transformation are unclear, so are many of the mechanisms by which known anticancer agents exert their effect. Most of these agents are small molecule chemicals that must be administered to patients via a parenteral infusion or bolus injection. Clinical complications following parenteral administration of chemotherapeutics have been documented and extra patient care (with the attendant cost) is considered essential for these patients. As a result, some of the recent efforts to discover chemotherapeutics have focused on finding agents that remain active following oral administration.

### SUMMARY

The present invention relates to (but is not limited to) methods of using protease inhibitors, including ritonavir (Norvir), lopinavir (Kaletra), or amprenavir (Agenerase) (or combinations thereof), for treating cancer in certain patients (e.g., patients who are HIV-negative or patients who are HIV-positive but do not have an AIDS-related cancer, such as Kaposi's sarcoma). The term “patient,” as used herein, excludes HIV-positive patients who have or who are being treated for Kaposi's sarcoma. In addition to the three protease inhibitors noted above, patients can be treated with indinavir (Crixivan), nelfinavir (Viracept), and saquinavir (Invirase and Fortovase), or with combinations of any of these protease inhibitors, with or without additional pharmaceutical agents (e.g., other anti-cancer agents, anti-nausea agents, or anti-pain medications)). Formulations (e.g., physiologically acceptable compositions formulated for administration by a parenteral or oral route) containing one or more of these protease inhibitors and, optinally, one or more additional therapeutic agents are within the scope of the present invention.

The cancer can be, but is not necessarily, one that is associated with EGF (e.g., one in which EGF or EGF receptors are elevated (e.g. overexpressed) or overly active)). Examples of such cancers include cancers of the pancreas, lung (e.g., non-small cell lung cancer (NSCLC) including adenocarcinomas, squamous, bronchoalveolar, and large cell cancers), breast, head and/or neck (including squamous cell carcinoma of the head and neck (SCCHN), prostate, colon, stomach, ovary, bladder, kidney (or renal system) and brain. The methods of the invention can be applied to any cancer in which a significant number of clinical isolates (e.g., at least about 1 in 8 (e.g., about 1 in 8; 1 in 7; 1 in 6; 1 in 5; 1 in 4; 1 in 3; or 1 in 2) exhibit increased expression of the EGF receptor (which may be referred to below as EGFR). The cancer can be a glioma and any of the cancers treated can be ones that are refractory to chemotherapy. The cancer can also be one in which erbB2 is expressed or overexpressed. The cancer can also be a melanoma, a squamous cell skin cancer, or a leukemia (e.g., chronic lymphocytic leukemia (CLL), acute lymphoblastic leukaemia (ALL), acute myeloid leukemia, or hairy cell leukemia; regardless of subtype).

While the methods of the invention are not limited to those in which patients are treated by an agent (or agents) that exert(s) an effect through any particular mechanism of action, we do believe that m-calpain is upregulated in certain tumors and that upregulation of m-calpain correlates with upregulation of the EGF receptor. Accordingly, the present invention features methods of inhibiting cancerous cells (e.g., inhibiting their survival, growth, proliferation, ability to metastasize, or any other aspect of their nature that, when inhibited, confers a therapeutic benefit on the patient) by administering one or more agents that inhibit calpain (e.g., m-calpain; calpain is also referred to as a calcium-activated neutral protease (CANP) or as a calcium-dependent protease). These agents include, as noted above, ritonavir, lopinavir, amprenavir, indinavir, nelfinavir, and saquinavir, any of which, or any combination of which, can be administered with another anticancer agent (i.e., a chemotherapeutic agent) or therapy (e.g., radiation therapy or surgical ablation of a tumor or other growth) and/or with an agent that improves the ADME profile of the calpain inhibitor. We may refer to ritonavir, lopinavir, and amprenavir as agents (or compounds or molecules) of Formula I, Formula II, and Formula III, respectively.

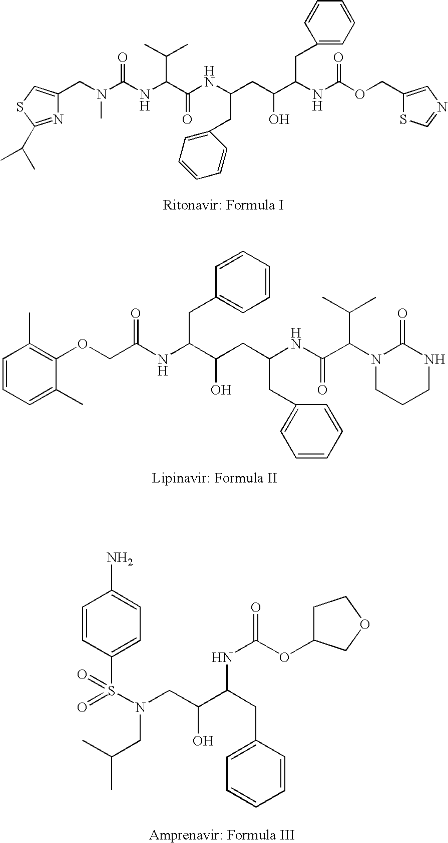

Other compounds that may be used include those of Formula IV or Formula V:

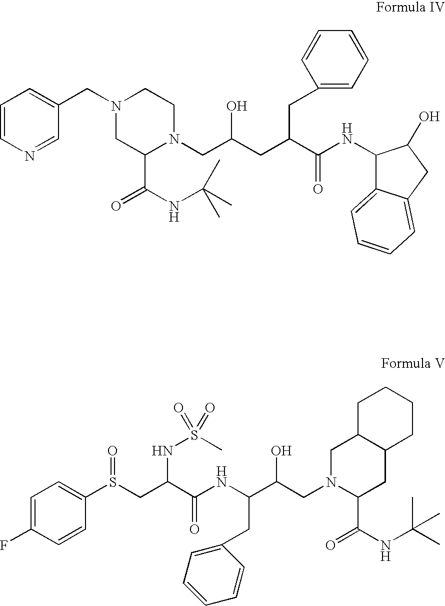

In addition, or alternatively, any of the methods of the invention and the compositions used to carry them out can include a pharmaceutically acceptable salt of a compound of Formula I, Formula II, Formula III, Formula IV, or Formula V. In addition, or alternatively, any of the methods of the invention and the compositions used to carry them out can include a prodrug of a compound of Formula I, Formula II, Formula III, Formula IV, or Formula V or an analog of any of Formulas I-V that retains sufficient ability to inhibit calpain to a therapeutically beneficial extent.

While treatment regimes are discussed further below, we note here that a calpain inhibitor (alone or in combination with other calpain inhibitors and/or other agents, as described herein) can be administered as a means of cancer prevention, and can be administered before, during, or after another anticancer agent or treatment. In some embodiments (e.g., when compositions comprising ritonavir (or another calpain inhibitor) are administered in conjunction with a Cox 2 inhibitor (or a less selective inhibitor that inhibits Cox 1 and Cox 2)), one can create a synergistic effect among the agents administered and thereby improve the outcome for a patient. For example, there may be synergy between paclitaxel and ritonavir (or other calpain inhibitors); between a Cox 2 inhibitor and ritonavir (or other calpain inhibitors); and between NFκB and ritonavir (or other calpain inhibitors) (synergism is not, however, required). These compositions are within the scope of the present invention. In particular embodiments, ritonavir, lopinavir, or amprenavir (or a combination thereof) are administered in combination with (i.e., before, during, or after) administration of a cytotoxic agent (a term that encompasses chemotherapeutics as well as other agents (see below)), a pain relief agent (e.g., a nonsteroidal anti-inflammatory drug such as celecoxib, or rofecoxib), an antinausea agent, or an anticancer agent other than ritonavir, lopinavir, or amprenavir (e.g., paclitaxel, docetaxel, doxorubicin, daunorubicin, epirubicin, fluorouracil, melphalan, cis-platin, carboplatin, cyclophosphamide, mitomycin, methotrexate, mitoxantrone, vinblastine, vincristine, ifosfamide, teniposide, etoposide, bleomycin, leucovorin, cytarabine, dactinomycin, interferon alpha, streptozocin, prednisolone, irinotecan, sulindac, 5-fluorouracil, capecitabine or procarbazine), an inhibitor of P-glycoprotein (e.g., ketoconazole), an inhibitor of an EGF receptor (e.g., an antibody that specifically binds and antagonizes an EGF receptor, such as antibody C225, antibody ADX-EGF, Iressa ZD1839, Tarceva OS1ZZ4, C1-1033, GW 572016, or EKB569; anti-EGF receptor antibodies can be humanized by methods known in the art; other inhibitors include antisense oligonucleotides or small inhibitory RNA (siRNA) molecules that specifically bind EGF receptor-encoding nucleic acid sequences), an inhibitor of erbB2, or a proteasome inhibitor (e.g., VELCADE™). Where an antibody is employed, it can be a humanized antibody, an antigen-binding fragment of an antibody (e.g., an Fab or F(ab′)₂ fragment) or a single-chain antibody.

The invention also features methods of predicting the sensitivity of a cancerous cell (e.g., a cell within a cancer cell line (cells within these lines are widely accepted as imperfect but useful models of various cancers) or a cell obtained from a patient) to a calpain inhibitor (e.g., as listed above (e.g., ritonavir, lopinavir, or amprenavir) or a composition containing same) by determining the relative amount of either m-calpain (or its level of activity) or an EGF receptor (or its level of activity) in the cell. The methods can be carried out simply by examining the expression of m-calpain and/or an EGF receptor in the cancerous cell (alternatively, or in addition, activity can be assessed); cells with elevated levels of either of these molecules (or both) will be more resistant to calpain inhibitors whereas cells with lower levels of m-calpain or EGF receptor will be more sensitive to calpain inhibitors. This information can be used to predict whether a given treatment regime (e.g., ritonovir, ritonovir in combination with lopinovir, or other protease inhibitors) will be effective in treating a particular cancer or a particular patient. For example, when carried out with a cancer cell line, one can determine whether the cell line is responsive and predict whether a patient who has a cancer (e.g., a cancer of the same type) will respond to treatment with a composition comprising a calpain inhibitor by: (a) providing cells from a cancer cell line (preferably, a cell line that is a model of the type of cancer the patient has) and (b) determining the level of expression or activity of m-calpain or the level of expression or activity of an EGF receptor in cells of the cell line. A level of m-calpain or of an EGF receptor that is higher than the level of expression or activity of m-calpain or an EGF receptor, respectively, in a reference cell, or population of reference cells (e.g., a cell or cells obtained from an individual (or individuals) having the same cancer as the patient being tested), indicates that the patient is expected to be less sensitive (or less responsive) to a treatment described herein (or to require more aggressive or prolonged treatment). Conversely, a level of m-calpain or of an EGF receptor that is lower than the level of expression or activity of m-calpain or an EGF receptor, respectively, in a reference cell, or population of references cells (e.g., a cell or cells obtained from an individual (or individuals) having the same cancer as the patient being tested), indicates that the patient is expected to be more sensitive (or more responsive) to a treatment described herein (or to require less aggressive or shorter treatment). The same method can be carried out with cancerous cells obtained from a patient (e.g., cells obtained from biopsy tissue). Expression or activity of the cellular components described here (e.g., m-calpain and the EGF receptor) can be carried out by methods routinely used by molecular biologists (e.g., Northern blot analysis, RNAse protection assay, a PCR-based assay (e.g., RT-PCR), with a DNA microchip, or by Western blot or other antibody-based assay). Accordingly, the methods of the invention can include the step of identifying a patient amenable to treatment.

Similarly, one can carry out methods of selecting a treatment regime for a patient by providing cancerous cells (those of an established cell line or those obtained from a patient who has cancer), exposing the cells (in vivo (e.g., in an animal model) or in cell culture) to at least two different compositions comprising a calpain inhibitor, and determining which, if any, of the compositions is most effective against those cells (as evidenced by, for example, the ability of the composition to kill the cells, reduce their motility, or reduce the rate at which they grow or proliferate). The compositions tested can be identical except in the concentrations of the components they contain, and they can include any of the calpain inhibitors, other agents, or formulations described herein. The methods in which a calpain inhibitor is administered to a patient can include the step of testing the inhibitor-containing composition as described here.

The methods described herein may be more advantageous than existing methods (e.g., methods of treating cancer) because the compounds of the invention, in some formulations, may have greater chemical or pharmacological stability, greater potency, different resistance profiles, different selectivity profiles, and decreased side effects.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A and 1B are photomicrographs depicting an immunohistochemical (IHC) study of a ductal breast cancer specimen using affinity purified UMC antibody.

FIGS. 2A and 2B depict pictures of gel shift assays that demonstrate co-migration of m-calpain with the 26S bovine proteasome on a glycerol gradient.

FIG. 3 is a bar graph showing a dose dependent decrease of inomycin activated m-calpain with ritonavir.

FIG. 4 depicts in vitro IC₅₀ data for amprenavir in A549, H460 and H23 cells as determined by percent of control cell survival.

FIGS. 5A-C depict an IHC analysis of the effect of ritonavir on cell differentiation in tongue epithelium of mice. FIG. 5A depicts a control mouse treated with Tween 80 vehicle. FIG. 5B depicts a mouse treated with 40 mg/kg ritonavir, having a peak serum ritonavir level of 12 μM. FIG. 5C depicts a mouse treated with 40 mg/kg ritonavir, having a peak serum ritonavir level of 67 μM.

FIGS. 6A-6D depict ritonavir animal data from the MDA-MB-231 xenograft model. FIG. 6A depicts the reduction in tumor growth of two of three mice with administration of ritonavir. FIG. 6b depicts tumor growth of three mice with administration of vehicle only. FIG. 6C depicts change in tumor weight at days 1, 6, 9, and 13 for control mice and mice administered ritonavir. FIG. 6D depicts change in animal weight at days 1, 3, 6, 10, and 13 for control mice and mice administered ritonavir.

FIG. 7 depicts the synergistic effect in IC₅₀ with the co-administration of ritonavir and docetaxel with MDA-231 cells as measured by the change in percent cell survival.

FIGS. 8A-8G depict the synergystic effect of ritonavir with VELCADE™ in H460, A459, H23, H522 and Caco 2 cells as measured by the reduction in percent cell growth inhibition IC₅₀s.

### DETAILED DESCRIPTION

m-calpain activation is an early event in neoplasia, resulting in inhibition of c-Cbl and loss of the ubiquitin ligase activity that inhibits the EGF receptor and ErbB2 receptors. In the event of neoplastic transformation, activation of the EGF or ErbB2 receptor leads to ERK-mediated activation of m-calpain by phosphorylation on Serine 50, independent of Ca²⁺. m-Calpain is proposed to be an amplifier of the receptor tyrosine kinase. Therapeutic inhibition of calpain can be achieved by small molecule inhibitors, such as ritonavir. m-calpain activity may be not only an amplifier of EGF receptor and ErbB signaling, but also a necessary activity for maintaining adequate levels of these receptors to promote cancer cell survival and proliferation.

Calpain has been implicated in the development and progression of cancer. Calpastatin over-expression or knockout of the regulatory small subunit of m-calpain, CAPN4, suppresses morphologic transformation and anchorage of independent cell growth resulting from the activity of v-src (Carragher et al., Mol. Cell Biol. 22:257-269 (2002)). Additionally, calpastatin over-expression in p53 wild type cells represses the progression of v-src transformed cells through the G1 checkpoint of the cell cycle. Accordingly, calpain activation by oncogenes may be an essential event early in carcinogenesis. Calpain also cleaves wild type p53, while mutant p53 is relatively resistant (Kubbutat et al., Mol. Cell Biol. 17:460-468, 1997). This finding suggests that calpain activation early in carcinogenesis may allow proteolysis of p53, abrogating the function of p53 without mutation. It was also observed, by IHC detection of calpain cleavage products of α-fodrin, that the earliest pancreatic intraepithelial neoplasia demonstrate calpain activation.

Additional evidence for an early role for calpain in carcinogenesis comes from microarray gene expression studies of the NCI-60 database of cancer cell lines (see, e.g., genome-www.stanford.edu/nci60/search.shtml). A survey of the NCI-60 lines shows that m-calpain is upregulated in half or more of the cancer cell lines (Ross et al., Nat. Genet. 24:227-235, 2000). Moreover, there is a strong correlation between m-calpain and EGFR expression levels. In over 80% of the tumors, the expression was concordant, the genes being either up or down in expression, in tandem. This strongly suggests that m-calpain and EGFR are co-regulated. There was no similar correlation between μ-calpain, the other ubiquitous calpain isoform, which lacks the ERK phosphorylation site, and EGFR gene expression. Of interest, the breast cancer cell lines of the NCI-60 database appeared to have tandem high or low m-calpain/EGFR expression, confirmed by analysis of gene expression microarray data published by Ross et al. (Nat. Genet. 24:227-235, 2,000). See the Examples below.

Calpain Inhibitors

Ritonavir, lopinavir, amprenavir, and other protease inhibitors have been approved by the Food and Drug Administration and can be formulated and used, alone or in various combinations, in the methods described herein.

Ritonavir Inhibits Calpain and not the Proteasome in Intact Cells: In addition to, or instead of the ritonavir shown in formula I, stereoisomers conforming to formula I can be used in the methods described herein, for example, the compound shown below.

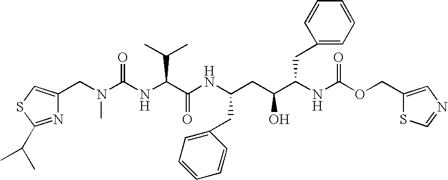

Identification of the intracellular target for ritonavir is important for the development of this drug as an anti-cancer agent. Target identification assists the development of pharmacodynamic markers for drug efficacy as well as the development of strategies for dosing and the identification of potentially synergistic drugs. It has been proposed that ritonavir inhibits the proteasome, based on inhibition of cleavage of the substrate suc-LLVY-AMC by the chymotryptic activity of the 20S proteasome (Gaedicke et al., Cancer Res. 62:6901-6908, 2002; Andre et al., Proc. Natl. Acad. Sci. USA 95:13120-13124, 1998), which is contained in the β5, or X, subunit. This active site is also specifically blocked by the inhibitor lactacystin, which interacts with this subunit and no other protease in intact cells, as demonstrated by in vivo labeling experiments with ³H-lactacystin (Fenteany et al., Science 268:726-731, 1995). Inconsistent with the hypothesis that ritonavir blocks this subunit are the observations that ritonavir does not inhibit cleavage of carbobenzoxyl-glycyl-glycl-leucyl-AMC (ZGGL-AMC), which is a substrate of the chymotryptic active site of the proteasome (Andre et al., Proc. Natl. Acad. Sci. U.S. A. 95:13120-13124, 1998). Also inconsistent with this hypothesis is the observation that ritonavir does not inhibit translocation of NF-κB to the nucleus (Pati et al., Blood 99:3771-3779, 2002). The results of studies aimed at reconciling these observations, and establishing ritonavir as a calpain inhibitor, are presented in the Examples.

Other Calpain inhibitors: Other compounds useful in the methods described herein include lopinavir, amprenavir, indinavir, nelfinavir, and saquinavir. In addition to, or instead of the inhibitors recited above, stereoisomers conforming to formulas II-V can be used in the methods described herein, for example, the compounds shown below.

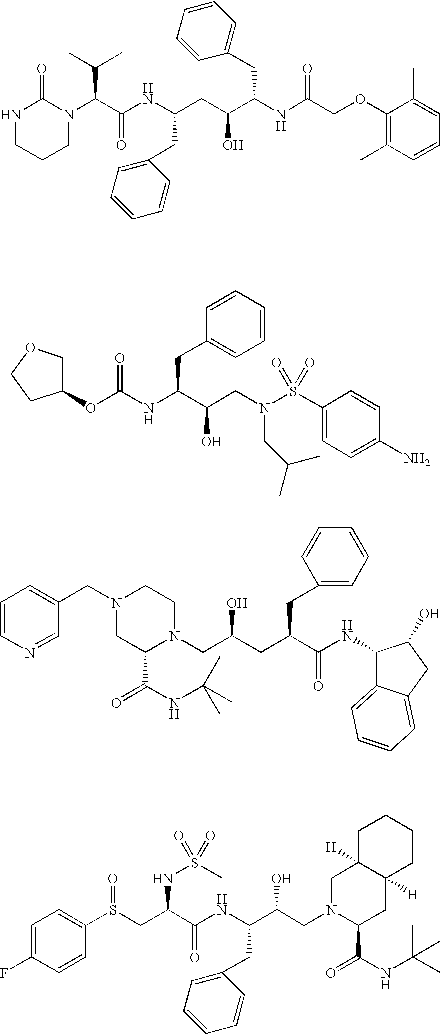

Any of the methods of the invention (including the therapeutic or prophylactic methods) can be carried out with a single calpain inhibitor or combinations of such inhibitors (a “monotherapy”), but combination therapies in which one or more calpain inhibitors are administered in conjunction with biologics, radiation or cytotoxic chemotherapy are also within the scope of the present invention. For example, ritonavir, lopinavir, and amprenavir (or combinations thereof) can be administered in conjunction with other agents, and methods of treating cancer (or reducing the likelihood of its occurrence or recurrence) by such treatment regimes are within the scope of the invention. For example, the formulations described above (e.g., ritonavir or ritonavir and lopinavir) can be administered in combination with agents that interfere with the mechanism by which NF-κB promotes chemoresistance (e.g., one can express the IκB a super-repressor of NF-κB). Alternatively, or in addition, a composition that includes ritonavir can be administered in conjunction with a Cox 2 inhibitor. While the methods of the invention are not limited to the use of compositions that function by any particular cellular mechanism, we note that there is evidence that PKA negatively regulates m-calpain by direct phosphorylation at SerThr, 369, 370, while ERK is activating by phosphorylating Ser50. Because the EP1 and 3 receptors for PGE2 can down-regulate cAMP and up-regulate Ca²⁺, we propose that, by blocking PGE2 production and EP1 and 3 signaling, Cox 2 inhibitors will promote up-regulation of cAMP and down-regulation of Ca²⁺, thereby inhibiting calpain and synergizing with ritonavir-containing compositions. The EGF receptor tyrosine kinase inhibitors including, but not limited to, Iressa (ZD1839), Tarceva (OSI-774), and EKB 569 can also be administered with (and may synergize) ritonavir-containing compositions. The same is true for EGF receptor antibodies, such as C225, or other EGFR inhibitors (the sequence of the receptor, including the sequence of the human EGFR is available and can be used to make antisense oligonucleotides or double-stranded RNAs (e.g., siRNAs) that inhibit EGFR expression when administered to cells by methods known in the art for inhibiting gene expression with such compounds) and Src inhibitors that activate EGFR. In addition, MEK, Ras, Grb, SOS, Raf and ERK inhibitors (e.g., small molecule or antisense inhibitors) can be administered in conjunction with (and may synergize) ritonavir-containing compositions.

Calpain inhibitors can also be administered in combination with a cytotoxic agent. Examples of cytotoxic agents include anti-microtubule agent, a topoisomerase I inhibitor, a topoisomerase II inhibitor, an anti-metabolite, a mitotic inhibitor, an alkylating agent, an intercalating agent, an agent capable of interfering with a signal transduction pathway, an agent that promotes apoptosis or necrosis, and radiation.

In yet other formulations, a calpain inhibitor such as ritonavir can be combined with (and used to treat patients or screen their cells, as described above) a proteasome inhibitor such as VELCADE™ (bortezomib). See the data presented in the Examples below, indicating that VELCADE™ enhances the cytolytic effects of ritonavir. More specifically, ritonavir, lopinovir, and/or amprenavir (or various combinations thereof) can be administered in conjunction with proteasome inhibitors, such as VELCADE™ (bortezomib). If necessary, or desirable, the activity of VELCADE™ (bortezomib) can be determined by examining the activity of the 20S proteasome. Resveratrol and parthenolide, IκB kinase inhibitors (Holmes-McNary et al., Cancer Res. 60:3477-3483, 2000; Hehner et al., J. Immunol. 163:5617-5623, 1999), are also better NF-κB inhibitors than the super-repressor, and thus useful compounds for co-administration with ritonavir or other calpain (e.g., m-calpain) inhibitors.

The compositions of the invention (and the methods in which those compositions are used) may also be defined by the agents that are excluded. For example, the methods of the invention (e.g., a method for treating an HIV-negative patient who has cancer (e.g., a cancer described herein)) can include use of a pharmaceutically effective (or physiologically acceptable) composition that includes a compound of Formula I (or a pharmaceutically acceptable salt, prodrug, or analog thereof) but that excludes, independently, a compound of Formula II, Formula III, Formula IV, or Formula V (or a pharmaceutically acceptable salt, prodrug, or analog of Formula II-V). For ease of reading, we will not always repeat the caveat that a compound that is a pharmaceutically acceptable salt, prodrug, or analog of a given Formula can be used in place of a compound of that formula. Such substitutions are to be assumed unless otherwise specifically noted. In another embodiment, the methods of the invention (e.g., a method for treating an HIV-negative patient who has cancer) can include use of a composition (an effective and/or acceptable composition as noted above) that includes a compound of Formula III but excludes a compound of Formula I, II, IV, or V. In another embodiment, the methods of the invention can include the use of a composition (as above) that includes a compound o Formula IV but excludes a compound of Formula I, II, III, or V or of a composition that includes a compound of Formula V but excludes a compound of Formula I, II, III, or IV.

Where a compound of Formula II-V is combined with a compound of Formula I, the amount, by weight, of the compounds of any of Formulas II-V can be about four times (e.g., about three, four, or five times) greater than the amount, by weight, of Formula I.

Any of the compositions of the invention, or used in the methods of the invention, can include a carrier, excipient, or diluent (e.g. a physiologically acceptable saline solution).

Patients Amenable to Treatment

The methods of the invention can be used to treat patients who are HIV-negative or patients who are HIV-positive but do not have an AIDS-related cancer, such as Kaposi's sarcoma. Notably, the methods can be applied to patients who have been diagnosed as having a cancer or other proliferative disorders as well as to patients who are only considered at risk for such disorders (either initially or as a recurrent event). That is, the methods of the invention encompass chemotherapy and chemoprevention. For example, the methods of the invention can be applied to a patient who has an intraepithelial neoplasia or dysplasia to prevent progression to cancer. For example, the compositions described herein can be administered to patients suspected of having (or who have had) a proliferative disorder affecting cells within the pancreas, lung, breast, head and neck, prostate, colon, stomach, ovary, bladder, kidney (or renal system), blood, skin, and brain. The disorder may also be one involving glia. Because ritonavir can be tolerated on a daily basis as a non-cytotoxic agent, it can be used as a chemopreventative agent, either formulated alone (or essentially alone (e.g., as the primary active agent in a composition)) or when formulated with or administered with other agents (e.g., lopinavir). As noted above, the compositions useful in therapeutic or prophylactic regimes can include, or can be administered in conjunction with, agents other than calpain inhibitors. For example ritonavir (or a combination of ritonavir and lopinavir) can be administered with low dose retinoids and/or Cox 2 inhibitors (to treat or prevent, for example, head and neck cancer) or in combination with tamoxifen and/or aromatase inhibitors (to treat or prevent, for example, a breast cancer or disorder).

Examples of disorders in which cells proliferate or differentiate in an undesirable way (including cells that become malignant) include disorders of the following tissues: pancreas, lung, breast, head and neck, prostate, colon, stomach, ovary, bladder, kidney (or renal system), blood, skin, and brain. The disorder may also be one involving glia.

Disorders of the breast include, but are not limited to, proliferative breast disease including, for example, epithelial hyperplasia, sclerosing adenosis, and small duct papillomas; tumors, for example, stromal tumors such as fibroadenomas, phyllodes tumors, and sarcomas, and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; and miscellaneous malignant neoplasms. Disorders in the male breast include, but are not limited to, gynecomastia and carcinoma.

Examples of proliferative or differentiation disorders of the colon include, but are not limited to, non-neoplastic polyps, adenomas, familial syndromes, colorectal carcinogenesis, colorectal carcinoma, and carcinoid tumors. Other disorders include colorectal neoplasia and familial cancer syndromes.

Any of the methods of treating a patient can be applied where the patient has a cancer associated with resistance to known anticancer drug regimes (e.g., wherein the cancer comprises cells that express a P-glycoprotein (MDR), a multidrug resistance-associated protein (MRP), or a breast cancer resistance protein (BCRP).

Formulations and Routes of Administration

Whether a patient is treated before or after a diagnosis has been made, the compositions of the invention will be delivered in therapeutically effective amounts (i.e. amounts that confer a beneficial effect on the treated subject). The therapeutic effect may be objective (i.e., measurable by some test or marker) or subjective (i.e., the patient subject gives an indication of or feels an effect). The dose level of the compounds of Formulas I-V and the frequency of dosage of the specific combination, will vary depending on a variety of factors including the potency of each specific compound employed, the metabolic stability and length of action of that compound, the patient's age, body weight, general health, sex, diet, mode and time of administration, rate of excretion, drug combination, the severity of the condition to be treated, and the patient undergoing therapy.

The compounds of this invention can be synthesized using conventional techniques. Advantageously, these compounds are conveniently synthesized from readily available starting materials. In general, the compounds of the formulae described herein are conveniently obtained via standard organic chemistry synthesis methods, including those methods illustrated in the schemes and the examples herein. Alternatively, many of the agents used in the methods of the present invention are commercially available. For example, protease inhibitors are commercially available. Amprenavir (brand name Agenerase) can be purchased from GlaxoSmithKline; indinavir (brand name Crixivan) can be purchased from Merck & Co.; lopinavir (brand name Kaletra) and ritonavir (brand name Norvir) can be purchased from Abbott Laboratories; nelfinavir (brand name Viracept) can be purchased from Agouron Pharmaceuticals; and saquinavir (hard gel cap brand name Invirase; soft gel cap; brand name Fortovase) can be purchased from Hoffman LaRoche Laboratories.

For use in medicine, the salts of the agents described herein (e.g., compounds of Formulas I-V) will be pharmaceutically acceptable salts. Other salts may, however, be useful in the preparation of the compounds (e.g., in preparation of compounds of Formulas I-V), or of their pharmaceutically acceptable salts. Suitable pharmaceutically acceptable salts of the compounds of Formulas I-V include acid addition salts which may, for example, be formed by mixing a solution of the compound according to the invention with a solution of a pharmaceutically acceptable acid. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Suitable pharmaceutically acceptable salts of calpain inhibitors, including those of the Formulas I-V, also include salts derived from appropriate bases include alkali metal (e.g., sodium), alkaline earth metal (e.g., magnesium), or ammonium and N-(alkyl)4+ salts. This invention also envisions the quaternization of any basic nitrogen-containing groups of the compounds disclosed herein. Water or oil-soluble or dispersible products may be obtained by such quaternization.

Compounds of Formulas I-V, or other calpain inhibitors, may also form solvates such as hydrates, and the invention also extends to these forms.

As used herein, the compounds of this invention, including the compounds of formulae described herein, are defined to include pharmaceutically acceptable derivatives or prodrugs thereof. A “pharmaceutically acceptable prodrug” means any pharmaceutically acceptable salt, ester, salt of an ester, or other derivative of a compound of this invention which, upon administration to a recipient, is capable of providing (directly or indirectly) a compound useful in the methods of this invention. Particularly favored derivatives and prodrugs are those that increase the bioavailability of the compounds of this invention when such compounds are administered to a subject (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein. Conventional procedures for the selection and preparation of suitable prodrug derivatives are described, for example, in Design of Prodrugs, H. Bundgaard (Ed.), Elsevier Press, 1985.

The compounds of this invention may be modified by appending appropriate functionalities to enhance selective biological properties. Such modifications are known in the art and include those which increase biological penetration into a given biological compartment (e.g., blood, the lymphatic system, or the central nervous system), increase oral availability, increase solubility to allow administration by injection, alter metabolism and alter rate of excretion.

The compounds of the formulae delineated herein can be administered to a patient, for example, in order to treat disease or disease symptoms. The compounds can, for example, be administered in a pharmaceutically acceptable carrier such as physiological saline, in combination with other drugs, and/or together with appropriate excipients.

As the skilled artisan will appreciate, lower or higher doses than those recited herein can be required. Specific dosage and treatment regimens for any particular patient will depend upon a variety of factors, including the activity of the specific compound employed, the age, body weight, general health status, sex, diet, time of administration, route of administration, frequency of administration, rate of excretion, drug combination, the severity and course of the disease, condition or symptoms, the patient's disposition to the disease, condition or symptoms, and the judgment of the treating physician.

Pharmaceutical compositions of this invention include a compound of the formulas described herein or a pharmaceutically acceptable salt thereof; an additional agent, such as a cancer agent, and any pharmaceutically acceptable carrier, adjuvant or vehicle. Alternate compositions of this invention include a compound of the formulaes described herein or a pharmaceutically acceptable salt thereof; and a pharmaceutically acceptable carrier, adjuvant or vehicle. Such compositions can optionally include additional therapeutic agents, including, for example an additional agent such as a pain relief agent (e.g., nonsteroidal anti-inflammatory drug (NSAID)), an additional cancer agent, or an antinausea agent.

The term “pharmaceutically acceptable carrier or adjuvant” refers to a carrier or adjuvant that can be administered to a patient, together with a compound of this invention, that does not destroy the pharmacological activity thereof and is nontoxic when administered in doses sufficient to deliver a therapeutic amount of the compound.

Pharmaceutically acceptable carriers, adjuvants and vehicles that can be used in the pharmaceutical compositions of this invention include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-b-cyclodextrins, or other solubilized derivatives can also be advantageously used to enhance delivery of compounds of the formulae described herein.

The pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient can be suspended or dissolved in an oily phase combined with emulsifying and/or suspending agents. If desired, certain sweetening and/or flavoring and/or coloring agents can be added.

Dosage levels of between about 0.001 and about 100 mg/kg body weight per day, alternatively between about 0.5 and about 75 mg/kg body weight per day, or any range in which the lower number is between 0.001 and 99.9 inclusive, and the upper number is between 0.002 and 100 inclusive and is higher than the lower number, mg/kg body weight per day, of the compounds described herein are useful in a monotherapy and/or in combination therapy for the prevention and treatment of disease. Typically, the pharmaceutical compositions of this invention will be administered from about 1 to about 6 times per day (e.g., at 10 mg-1000 mg/dose; or any range in which the lower number is an integer between 10 and 999 inclusive, and the upper number is an integer between 11 and 1000 inclusive that is higher than the lower number) or alternatively, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Given that some of the agents useful in the present invention are FDA-approved for other indications, present dosing scenarios can be used to guide those of ordinary skill in the art who wish to practice the present invention. The estimated dose of ritonavir is 600 mg, given orally, BID. Higher doses may be appropriate. The dose of celecoxib is 400 mg BID. Here again, higher doses may be appropriate.

The information in the table below represents a dosing recommendation for administering lopinavir to children. Each row represents a recommendation for a given weight range, and the dose in parentheses is recommended if either nevirapine or efavirenz are used in combination with lopinavir. Those of ordinary skill in the art can use this information, and information like it, to optimize dosing schedules and amounts for patients to be treated in accordance with the methods described herein.

| Weight | |
|---|---|
| (kg) | Amount of lopinavir oral solution |
| 7 to 10 kg | 1.25 ml (1.5 ml) twice a day |
| 10 to 15 kg | 1.75 ml (2.0 ml) twice a day |
| 15 to 20 kg | 2.25 ml (2.5 ml) twice a day |
| 20 to 25 kg | 2.5 ml (3.25 ml) twice a day |
| 25 to 30 kg | 3.0 ml (4.0 ml) twice a day |
| 30 to 40 kg | 3.5 ml (4.5 ml) twice a day |
| 40 to 50 kg | 5 ml or three capsules twice a day (no dose adjustment |
| | for nevirapine or efavirenz) |
| over 50 kg | 5 ml or three capsules (6.5 ml or four capsules) twice a |
| | day |

When the compositions of this invention include a combination of a compound of the formulae described herein and one or more additional therapeutic or prophylactic agents, both the compound and the additional agent should be present at dosage levels of between about 10% to 100%, and more preferably between about 10% to 80% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

Assays

The invention also features methods of predicting the sensitivity of a cancerous cell (e.g., a cell within a cancer cell line (cells within these lines are widely accepted as imperfect but useful models of various cancers) or a cell obtained from a patient) to a calpain inhibitor such as ritonavir, lopinavir, or amprenavir by determining the relative amount of either m-calpain (or its level of activity) or an EGF receptor (or its level of activity) in the cell. The methods can be carried out simply by examining the expression of m-calpain or EGF receptor in the cancerous cell (alternatively, or in addition, activity can be assessed); cells with elevated levels of either of these molecules (or both) will be more resistant to calpain inhibitors whereas cells with lower levels of m-calpain or EGF receptor will be more sensitive to calpain inhibitors. This information can be used to predict whether a given treatment regime (e.g., ritonovir, ritonovir in combination with lopinovir, or other protease inhibitors) will be effective in treating a particular cancer or a particular patient. For example, when carried out with a cancer cell line, one can determine whether the cell line is responsive and predict whether a patient who has a cancer (e.g., a cancer of the same type) will respond to treatment with a composition comprising a calpain inhibitor by: (a) providing cells from a cancer cell line (preferably, a cell line that is a model of the type of cancer the patient has) and (b) determining the level of expression or activity of m-calpain or the level of expression or activity of an EGF receptor in cells of the cell line. A level of m-calpain or of an EGF receptor that is higher than the level of expression or activity of m-calpain or an EGF receptor, respectively, in a reference cell, or population of reference cells, indicates that the patient is not likely to respond positively if treated with a composition comprising a calpain inhibitor. The same method can be carried out with cancerous cells obtained from a patient (e.g., cells obtained from biopsy tissue). Expression or activity of the cellular components described here (e.g., m-calpain and the EGF receptor) can be carried out by methods routinely used by molecular biologists (e.g., Northern blot analysis, RNAse protection assay, a PCR-based assay (e.g., RT-PCR), with a DNA microchip, or by Western blot or other antibody-based assay).

Similarly, one can carry out methods of selecting a treatment regime for a patient by exposing cancerous cells (those of an established cell line or those obtained from a patient who has cancer) by exposing the cells (in vivo (e.g., in an animal model) or in cell culture) to at least two different compositions comprising a calpain inhibitor and determining which, if any, of the compositions is most effective against those cells (as evidenced by, for example, the ability of the composition to kill the cells, reduce their motility, or reduce the rate at which they grow or proliferate). The compositions tested can be identical except in the concentrations of the components they contain, and they can include any of the calpain inhibitors, other agents, or formulations (all of which are within the scope of the present invention) described herein.

As implied above by the requirement for a “reference” cell, resistance to calpain inhibitors or other chemotherapeutic agents is a relative concept. We demonstrate with Caco 2 lines, engineered to have varying levels of m-calpain, that ritonavir sensitivity is inversely proportional to levels of m-calpain. Arbitrarily, we may define ritonavir resistant cell lines as those demonstrating an MTT assay IC₅₀ that is above the upper boundary of ritonavir Cmax plasma concentrations in patients, which is about 45 μM (Gatti et al.). Lines that are resistant are Caco 2, MDA-MB-231, SKBr-3, H522, H23, and H460. Lines that are sensitive include MCF7, T47D, MDA-MB-436SR (expressing the NF-κB super-repressor), Caco 2 C9 (expressing empty vector), Caco 2 2-3 and Caco 2 0.5-11 and AG549 (48 hour incubation with ritonavir).

A diagnostic test distinguishing tumors having higher m-calpain levels from tumors having lower m-calpain levels was developed by first raising rabbit antisera to the amino terminus of human m-calpain (sequence AGIAAKLAK (SEQ ID NO:______) and to the calpain cleavage product of α-fodrin (sequence QQEVY (SEQ ID NO:______)). The m-calpain antiserum recognizes intact m-calpain (80 kDa) (e.g., using a Western blot), while the α-fodrin antibody specifically recognizes the expected 150 kDa calpain breakdown product of a-fodrin (BDP). The first antiserum detects intact m-calpain activated by EGF receptor directed phosphorylation on serine 50 (Glading et al., J. Biol. Chem. 276:23341-23348). The second antiserum detects calpain activity.

The antisera include antiserum raised to the human m-calpain peptide NH₂-AGIAAKLAKGGG(C)-COOH (SEQ ID NO:______), which recognizes the uncleaved form of m-calpain (italics indicate linker; (C) is the linker cysteine). We hypothesize that this antiserum detects the isoform of m-calpain activated or inhibited by phosphorylation events, in contrast to Ca²⁺, since Ca²⁺ stimulation has been shown to result in trans-cleavage of this propeptide from m-calpain by mu-calpain (Tompa et al., J. Biol. Chem. 271:33161, 1996). The antisera also include antiserum raised to the human α-fodrin peptide NH₂-(c)GGGQQEVY-COOH (SEQ ID NO:______), which recognizes the calpain-cleaved amino terminal fragment of the protein (italics indicate linker). The cleavage is between tyrosine 1176 and glycine 1177.

These antisera were used to test four human squamous cell carcinomas of the head and neck (SCCHN), all of which are “base of tongue” cancers. Of the four cancers, three were strongly positive for EGF receptor, m-calpain, and BDP. The remaining cancer was weakly staining for all three antisera. Together, these results demonstrate a correlation between m-calpain levels and activity and EGF receptor levels, as in human cancer cell lines. These antisera were also used to test seven adenocarcinoma non-small cell lung cancers (NSCLC). Three of the NSCLC adenocarcinomas showed strong m-calpain and EGF receptor staining, and while four demonstrated weak m-calpain and EGF receptor staining. The BDP staining correlated well with the m-calpain stain, but the BDP antiserum also stained nuclei, (the reasons for this result were unclear).

The m-calpain stain also stained metaphase chromosomes, as predicted by Schollmeyer (Science 240:911-913). These findings, which demonstrate a correlation between m-calpain and EGF receptor levels in cancer cells, suggest that antisera to intact m-calpain, BDP and EGF receptor can be used to determine which SCCHNs and NSCLCs will be susceptible to calpain inhibitors (e.g., ritonavir, lopinaivir, and amprenavir). These antisera can also be used to determine breast and colon cancers susceptible to calpain inhibitors (e.g., ritonavir, lopinavir, and amprenavir; salts, prodrugs, analogs, or combinations thereof).

### EXAMPLES

### Example 1

### Ritonavir Purification and Formulation

Ritonavir liquid was purchased from a commercial pharmacy and was HPLC purified. Ritonavir solubility conditions for animal studies were determined empirically using oleic acid, cremophor, and ethyl alcohol, USP. A ratio of 80:12:8 of oleic acid, cremophor, and ethyl alcohol, USP resulted in excellent solubilization of the ritonavir. This formulation was used for mouse gavage experiments and was tolerated for about three weeks. For cell culture experiments, ritonavir was formulated in DMSO.

### Example 2

### Determination of Ritonavir Sensitivity of Human Breast Cancer and Melanoma Cell Lines

The m-calpain levels in five human breast cancer and melanoma cell lines were determined using Western blotting. These results were then normalized to the MCF7 cell line. (See the Table below.)

TABLE
Levels of m-Calpain and EGF Receptor in Cancer Cell Lines

| | m-calpain | EGF receptor |
|---|---|---|
| MCF7 | 1 | 1 |
| 435 | 2.88 | 0.809 |
| 231 | 8 | 1.42 |
| 436 | Similar to 231 | Higher than MCF7 |
| Caco 2 | Similar to 231 | Higher than MCF7 |

MCF7 and MDA-MB-435 (435) had relatively low m-calpain levels, whereas MDA-MB-231 (231), MDA-MB-436 (436) and Caco 2 had relatively high m-calpain levels. The MCF7 having an LD₅₀ 25 μM. 435 cells, having an LD₅₀=16 μM were more sensitive than the 436, 231 and Caco 2 lines, having LD₅₀s of 55 μM, 60 μm, and 70 μM, respectively.

MCF7 and 435 were susceptible to ritonavir treatment, while lines 436 and 231 were relatively resistant to ritonavir treatment. This data correlates with the peak plasma ritonavir concentrations ranging from 18.6-46 μM and trough concentrations ranging from 10.4-17.5 μM (Gatti et al., AIDS 13:2083-2089).

Additionally, sensitivity of 436 to ritonavir was increased with the expression of IκB a super-repressor of NF-κB, and is demonstrated in a shift in LD₅₀ from 55 μM to 40 μM. This finding is consistent with the implication of signaling pathways in the resistance of breast cancer cell lines to chemotherapy (Patel et al., Oncogene 19:4159-4169). Furthermore, this finding suggests a synergy between proteasome inhibitors, such as VELCADE™ (bortezomib) (Millennium Pharmaceuticals, Inc.) and calpain inhibitors when treating certain types of cancers.

### Example 3

### Proliferation Assays of Breast Cancer Cells Treated with Ritonavir

Proliferation assays were done by plating cells in a 96 well plate at 1 cells per well for the 231 cells and 5×10³ cells per well for the 435 cells. The cells were allowed to attach and grow for 24 hours. The cells were then exposed to ritonavir, added so that the final concentration of DMSO vehicle was 0.5%. The cells were allowed to grow for 24 hours and the number of viable cells was measured by MTT assay. The MDA-M-435 cells demonstrated an IC₅₀ of about 150 μM (averaging 2 experiments).

We note that the IC₅₀ is derived from MTT assays, while the LD₅₀ is derived from clonogenic assays. As the LD₅₀ for Caco 2 cells is about 50 μM, which is near the IC₅₀, that the IC₅₀ can potentially be used as a surrogate for the LD₅₀.

### Example 4

### EGF Receptor Expression in Colon Cancer Cells Treated with Ritonavir

Calpain inhibitor I or ritonavir was added to confluent Caco 2 cells in culture. The medium was not changed. After 24 hours, the cells were harvested by Triton X-100 lysis, and Western blot analysis was performed on the supernatant, which contains solubilized EGF receptor. The EGF receptor was down regulated by 20% in the case of treatment with ritonavir (20 μM). The EGF receptor was down regulated by 50% in the case of treatment with calpain inhibitor I (100 μM).

### Example 5

### Inhibition of Ionomycin M-Calpain Activity by Ritonavir in Caco 2a Cells

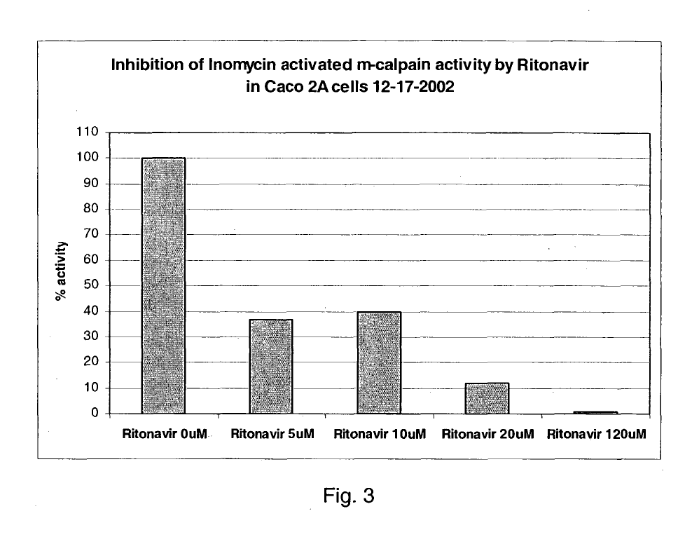

In order to determine whether ritonavir inhibits m-calpain at the cellular level, Caco 2A cells were treated with solutions of varying concentrations of Ritonavir (0, 5, 10, 20, and 120 μM). (The Caco 2a calls were treated and analyzed as in Example 4). The As depicted in FIG. 3, measurement of inomycin activated m-calpain, shows a dose dependent decrease when treated with ritonavir, thus providing evidence that ritonavir is an m-calpain inhibitor.

### Example 6

### Human Umbilical Vein Endothelial Cell (HUVEC) Tubule Formation Assay

Endothelial cell tubule formation in a fibrin clot is an analog of angiogenesis and was used to assay angiogenesis inhibitors (Brown et al., Lab. Investig. 75:539, 1996). HUVEC were coated on Cytodex™ microcarrier beads (Pharmacia) at a density of 30 HUVEC per bead, in a siliconized dish containing HUVEC complete medium, for 24 hours. Beads were suspended in a fibrin gel made by thrombin cleavage of fibrinogen, at an average of 20 beads per well of a 6-well plate, in 1.5 ml of medium. Vascular endothelial growth factor (VEGF) and bovine fibroblast growth factor (bFGF) were added to final concentrations of 60 and 20 ng/ml, respectively, with 10% fetal calf serum. The ritonavir was added to final concentrations between 0.01 and 100 μM. Tubules were counted at three days. Ritonavir inhibited the formation of new endothelial cell tubules with an IC₅₀ of 16 μM, by the same assay.

### Example 7

### HUVEC Cell Spreading Assay

HUVEC were preincubated with ritonavir (40 μM) and then spread for 1 hour on glass coverslips coated with fibronectin (10 μg/ml). Cells were fixed in 3.7% formaldehyde in PBS, blocked and stained with Oregon-green phalloidin 514 (Molecular Probes), and then digitally photographed using a Zeiss inverted fluorescence microscope equipped with a CCD camera. Cell areas were measured on the digital images using the Spot program (Diagnostic Imaging). For the cells treated with ritonavir, cell area was 4100±160 μm², 40 μM, indicating a 15% reduction of cell area at 1 hour of spreading. Under the same spreading conditions, the calpain inhibitor MDL 28, 170, 100 μM, blocked spreading by 21%. Ritonavir thus inhibits remodeling of the actin cytoskeleton associated with cell motility.

### Example 8

### Proliferation Assays of Non-Small Cell Lung Cancer Cells Treated with Amprenavir

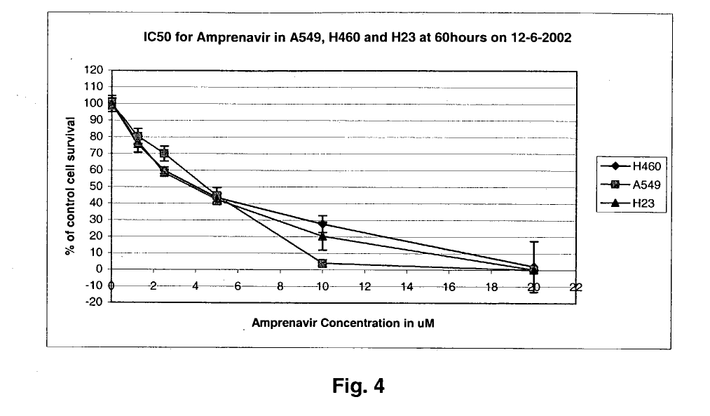

Three cancer cell lines (A549, H460, and H23) where treated with varying doses of amprenavir. A readout of cell survival was taken at 60 hours, providing the IC₅₀ for each of the three cell lines as depicted in FIG. 4. As seen in FIG. 4, all of the cell lines showed a dose response to amprenavir with IC₅₀s in the 4-5 μM range.

### Example 9

### Ritonavir Dosing in Mice

To develop a mouse model that allows the effect of ritonavir and ritonavir combinations on tumor growth to be measured, adequate serum levels of ritonavir (5-50 μM) as well as a pharmacodynamic marker for calpain inhibition are required. Oral gavage administration of the pharmaceutical liquid ritonavir (Norvir liquid, 80 mg/ml) in mice was ineffective for attaining adequate serum drug levels. Administration of 40 mg/kg body weight by this route resulted in peak ritonavir concentrations that were less than 1 μM. A vehicle control consisting of oleic acid, ethanol and cremaphor was toxic and resulted in death or weight loss in more than half of the animals, making this approach unsuitable.

Crystalline ritonavir was solublized in Tween 80 and administered by 50 μl ip injection in mice, resulting in peak serum drug levels between 12 and 67 μM. Trough drug levels were less than 0.1 μM, indicating a ritonavir half life of less than 3 h, a finding consistent with the rapid metabolism of ritonavir by mice (Granda et al., J. Pharmacol. Toxicol. Methods 40:235-239, 1998). These findings further indicate that ip administration circumvents the rapid first pass elimination of orally administered ritonavir in mice. IP ritonavir administration therefore makes

tumor response studies feasible in mice. Under these conditions, mice lost 0 to 5% of body weight and all mice remained viable after 10 days of treatment.

### Example 10

### Pharmacodynamic Measurement of Calpain Inhibition by Ritonavir in Mice

Mice were administered ritonavir as described in Example 8 and were sacrificed after 10 days of treatment with ritonavir. Ventral skin and tongue epithelial tissues were then examined for effects of ritonavir on differentiation and NFF score of the proliferating basal cells.

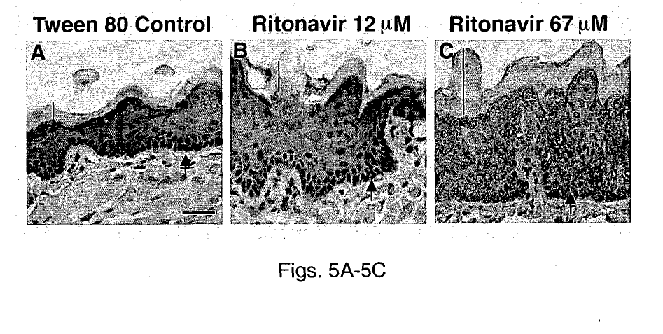

Differentiation of the stratum spinosum of tongue epithelium was affected, with a reduction of nuclear involution as well as an increase in the keratinized layer (FIGS. 5A-5C). Quantitatively, there is a marked decrease in the NFF score in the basal cell layer, from 91% for vehicle control to 34% for 12 μM ritonavir peak levels to 1% for 67 μM (Table VII). Previous observations of the proliferative rate of tongue compared to skin in mice indicate that tongue is a more proliferative epithelium (Hume, W. J. Cell Tissue Kinet. 19:195-203 (1986)). Findings of a higher NFF score for tongue, compared to ventral skin (91 compared to 44%) suggest a correlation between NFF score and epithelial proliferation (see the following Table).

TABLE
NFF score of calpain cleavage of α-fodrin in the basal cell
layer of tongue and ventral skin of ritonavir treated mice.*

| | NFF | NFF score (%) | NFF score (%) |
|---|---|---|---|
| | score (%) | ritonavir | ritonavir |
| | vehicle | (12 μM peak) | (67 μM peak) |
| Tongue | 91 | 34 | 1 |
| Ventral skin | 44 | 27 | 17 |

*Measurements were determined from paraffin block material stained by IHC with the PP1 antibody to the amino-terminal calpain cleavage product of α-fodrin, described in FIG. 5.
NFF score is determined by measuring the number of stained nuclei in the basal cell layer, based on a count of 100 nuclei.

FIGS. 5A-5C show the effect of ritonavir on differentiation. FIG. 5A shows mouse treated with Tween 80 vehicle control. Tongue tissue was fixed in formalin and paraffin embedded. The antiserum specific for the calpain-dependent α-fodrin cleavage, PP1, was used at 1:100 dilution. Competition with specific peptide blocked nuclear staining. The size bar is 60 μM. FIG. 5B shows mouse treated with 40 mg/kg ritonavir, resulting in a peak serum ritonavir level of 12 μM. FIG. 5C shows mouse treated with 40 mg/kg ritonavir, resulting in a peak serum ritonavir level of 67 μM. Arrows indicate basal layer nuclei. The vertical bar indicates maximum thickness of the keratinized layer in each image.

These data indicate that the NFF score can be used to determine the pharmacodynamic response of mice to ritonavir and indicate that we are able to attain adequate levels of ritonavir. These data also suggest that tongue or skin biopsies may be useful in subsequent clinical trials.

### Example 11

### Nude Mouse Tumor Xenograph Model

A mouse mammary fat pad model was used to determine whether ritonavir affects the growth rate of MDA-MB-435 human breast cancer xenografts in nude mice. An inoculum of 5×10⁵ MDA-MB-435 cells in 100 μl was injected into the mammary fat pad of each female animal. The tumors were allowed to grow for 31 days, at which time 16/29 animals had measurable tumors, the average size of which was 10-16 mm³. The mice were treated for 19 days with either vehicle (oleic acid:cremophor EL, ethanol USP, 80:12:8) or ritonavir, 10 or 20 mg/kg, by daily gavage. The tumor growth rate was reduced by 16% in the mice treated at 20 mg/kg, compared to the mice treated with vehicle. When one outlier tumor was removed from analysis of the animals treated with ritonavir at 10 mg/kg the tumor growth rate was reduced by 49% compared to the animals treated with vehicle. When an outlier tumor was removed from the analysis of the animals treated with ritonavir at 20 mg/kg, the tumor growth rate was reduced by 55% compared to the animals treated with vehicle.

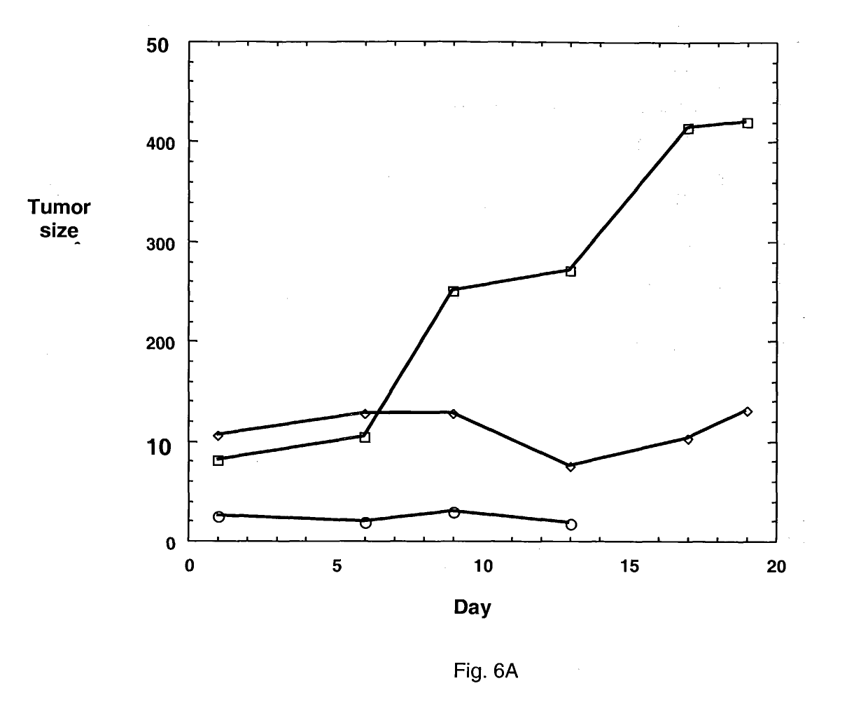

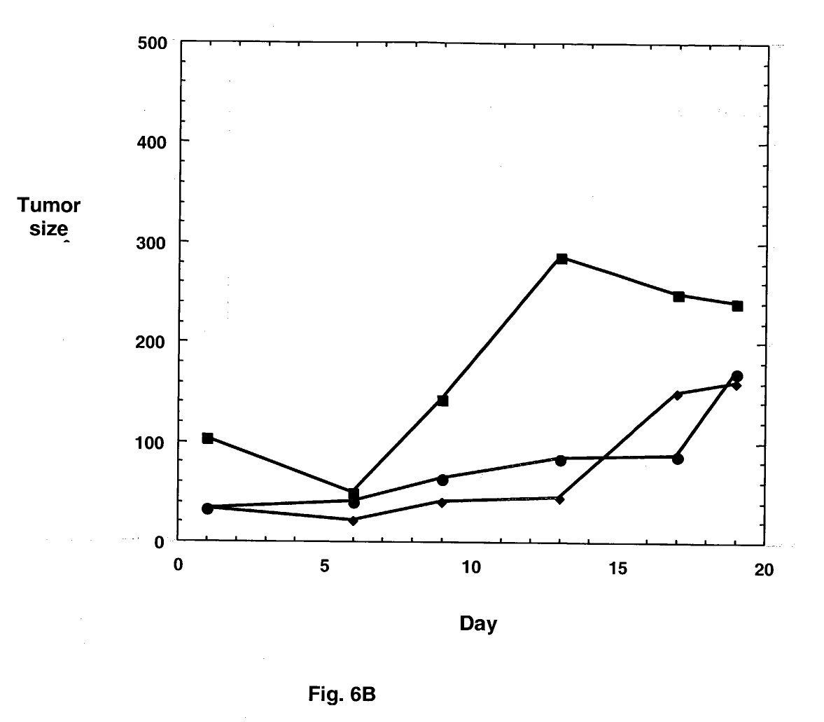

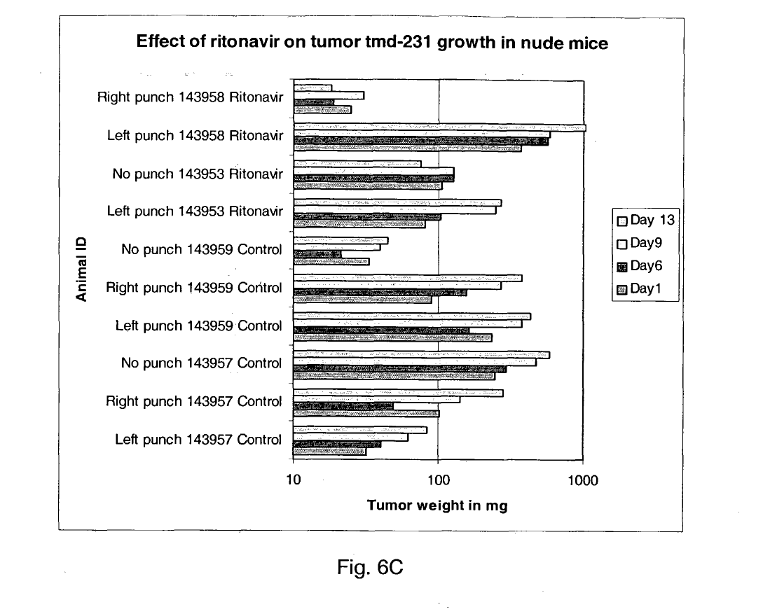

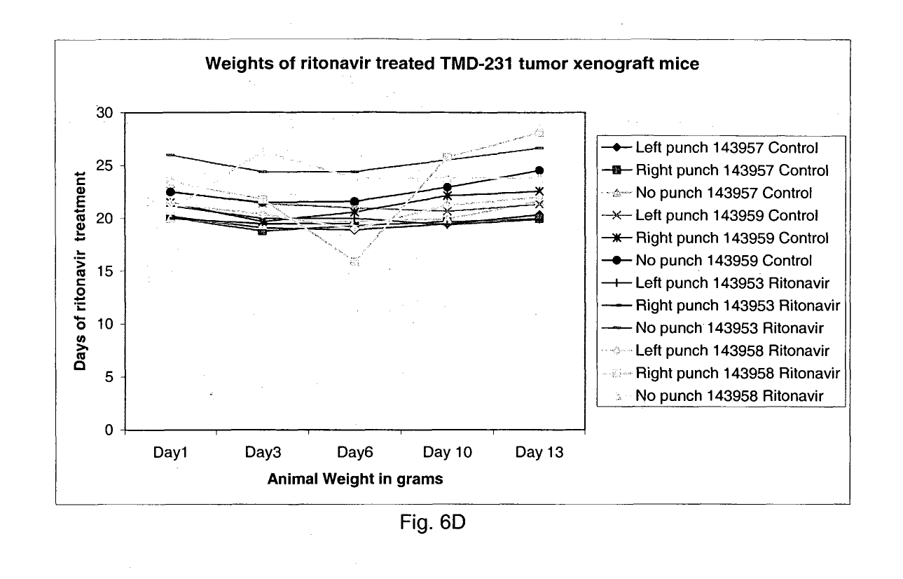

FIGS. 6A-6D depict the effect of ritonavir on three MDA-MB-231 human breast cancer tumors. The tumors were treated with 40 mg/kg of Ritonavir before their size was greater than 120 mm³. (Tumor size was measured by the method volume in cubic mm=length×[width]squared/2.) The tumors in this preliminary experiment took 7 weeks to grow and represent established tumors. (Established tumors are the preferred approach for testing the efficacy of anticancer drugs.) FIG. 6A demonstrates the efficacy of ritonavir treatment by showing that two tumors of three exhibited stable disease. FIG. 6B confirms the finding of 6A, showing that all three of the control tumors progressed.

Additional experiments, the results of which are depicted in FIGS. 6C and 6D, demonstrate that ritonavir treatment (40 mg/kg) be used to decrease the growth rate of tumors in nude mice. FIG. 6C depicts weight of the tumor when measured at days 1, 6, 9, and 13. FIG. 6D depicts weight of the animal when measured at days 1, 3, 6, 10, and 13. Together, FIGS. 6C and 6D show that a reduction in growth rate of the tumor in mice can be achieved without causing wasting of the mice.

### Example 12

### Synergy Between Ritonavir and Taxanes in Non-Small Cell Lung Cancer Cells

In order to determine the effectiveness of ritonavir combination therapies, ritonavir was administered with two members of the taxane family (paclitaxel and docetaxel) having known anticancer activity in the following non-small cell lung cancer cell lines: A459, H522, H460 and H23. As shown below in the three tables below, the combinations of Ritonavir with Pacliataxel and Ritonavir with Docetaxel showed synergistic effects in both cases.

TABLE
IC₅₀ Values of Taxanes and Ritonavir for NSCLC Cell Lines*

| | Ritonavir | Paclitaxel | Docetaxel |
|---|---|---|---|
| Cell Line | (μM) | (nM) | (nM) |
| A459 | 35 | 90 | 4.5 |
| H522 | 42.5 | 105 | 4.9 |
| H460 | 47.5 | 120 | 5 |
| H23 | 44 | 110 | 5.5 |

*IC₅₀ values were determined by MTT assay.

TABLE
IC₈₀ Values for Taxanes and Ritonavir and their
Combination in NSCLC Cell Lines*

| | | | | Paclitaxel/ | Docetaxel/ |
|---|---|---|---|---|---|
| Cell | Paclitaxel | Docetaxel | Ritonavir | Ritonavir | Ritonavir |
| Line | (nM) | (nM) | (μM) | (nM/μM) | (nM/μM) |
| A549 | >200 | >50 | 45 | 50/22.5 | 2.5/22.5 |
| H522 | >200 | >50 | 90 | 50/22.5 | 2.5/22.5 |
| H460 | >200 | >50 | 100 | 50/22.5 | 2.5/22.5 |

*IC₈₀ values were determined by MTT assay.

TABLE
Chou-Talalay Combination Index (CI) for Ritonavir,
Paclitaxel and Docetaxel for NSCLC Lines*

| Cell line | Ritonavir/Paclitaxel | Ritonavir/Docetaxel |
|---|---|---|
| A549 | 0.55 | 0.3 |
| H522 | 0.48 | 0.2 |
| H460 | 0.85 | 0.4 |

*Combination Index was calculated based on MTT assay and Chou-Talalay analysis using CalcuSyn software and averaged over an effective range of 0 to >0.90.

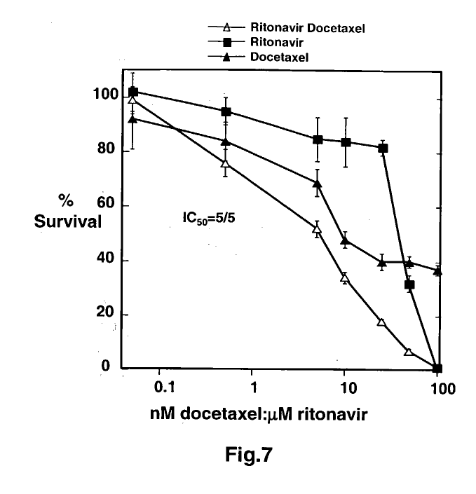

The synergy between ritonavir and docetaxel was also tested in the MDA-231 cell line (FIG. 7). Chou-Talalay isobologram analysis indicated a CI (cooperativity index) of 0.2, which is interpreted as strong synergy (Chou et al., Adv. Enzyme Regul. 22:27-55, 1984). This result suggests that combining docetaxel with ritonavir can make the tumors sensitive to low, clinically achievable doses of the drugs (FIG. 7). The MDA-231 cells were treated with ritonavir and/or docetaxel for 48 hours. The IC₅₀ for ritonavir was 60 mM and for docetaxel 10 nM. The drugs were tested together at the ratio of their IC₅₀ as single agents, over a 4-log range of concentrations. The isobologram IC₅₀ for the combination was 5 mM ritonavir/5 nM docetaxel. Viable cell number was measured by MTT assay. Similar results were observed for the lines MCF7, SKBR-3, and MDA-436 (Table XI). Remarkably, no synergy was observed for paclitaxel.

TABLE
Synergy of Ritonavir with Docetaxel

| | Ritonavir | Ritonavir |
|---|---|---|
| Cell line | IC₅₀ [μM] | Docetaxel CI |
| MCF7 | 25 | 0.2 |
| T47D | 12 | 5 |
| MDA-231 | 50 | 0.2 |
| SKBR-3 | 60 | 0.5 |
| MDA-436 | 40 | 0.2 |
| MDA-436LXSN | 42 | ND |
| MDA-436IκBSR | 26 | ND |

### Example 13

### Synergy Between Ritonavir and Other Agents in Caco 2 Cells

In order analyze ritonavir combination therapies for treatment of colon cancer, ritonavir was tested in Caco 2 cell lines with other known therapeutic agents. The results of ritonavir treatment with 5-FU, celecoxib, paclitaxel, and docetaxel in Caco 2 cell are depicted in the Table below. The data indicates that the most synergistic combination is ritonavir with docetaxel, where the least synergistic combination is ritonavir with celecoxib.

TABLE
Ritonavir Synergizes with Taxanes Paclitaxel and Docetaxel
and is Additive with 5-FU and Celecoxib

| | IC₅₀ | Drug + Ritonaivr, | Cooperativity |
|---|---|---|---|
| Agent | (μM) | IC₅₀ condition | Index (CI) |
| 5-FU | 28 | 3.5 + 22.5 | 1.0 |
| Celecoxib | 60 | 20 + 30 | 1.1 |
| Paclitaxel | 0.40 | 0.05 + 20 | 0.6 |
| Docetaxel | 0.005 | 0.0025 + 22.5 | 0.2 |
| Ritonavir | 45 | — | — |

### Example 14

### Synergy Between Ritonavir and VELCADE™ in Caco Cells

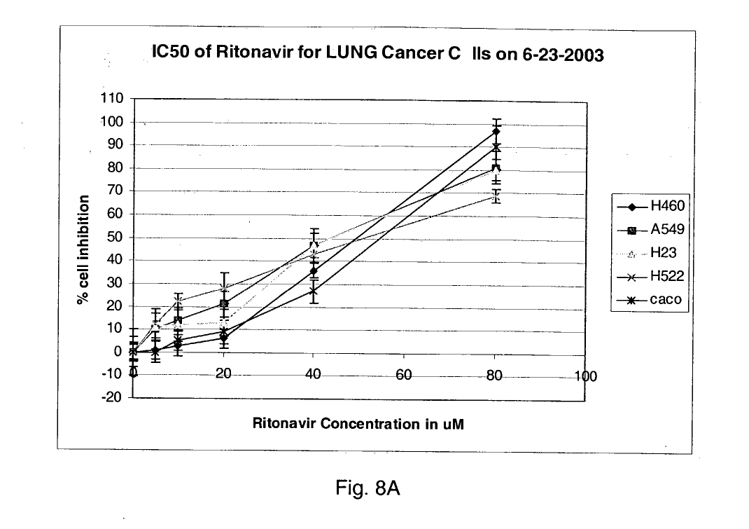

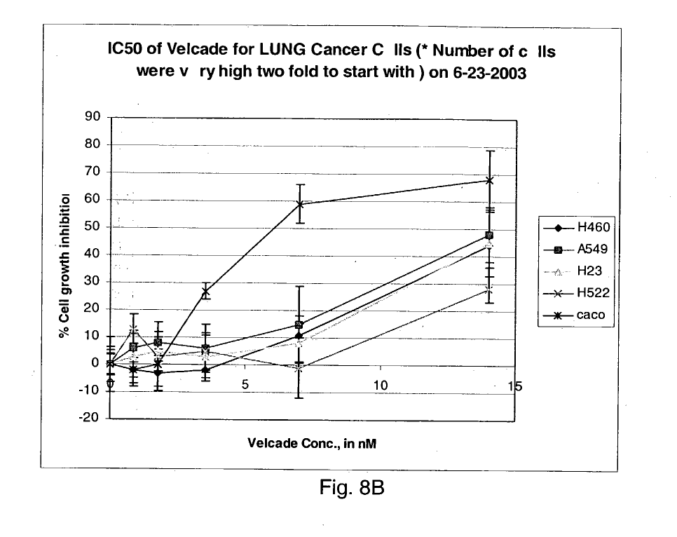

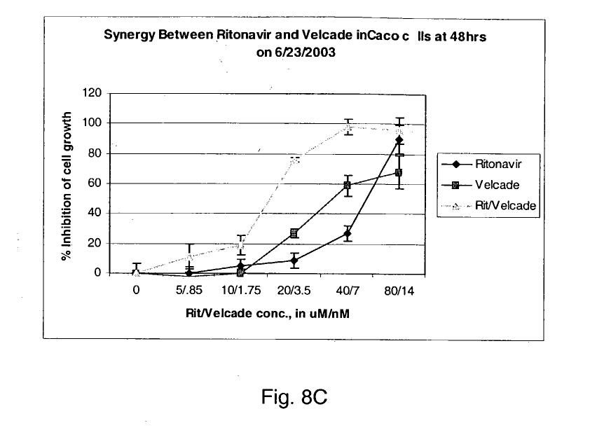

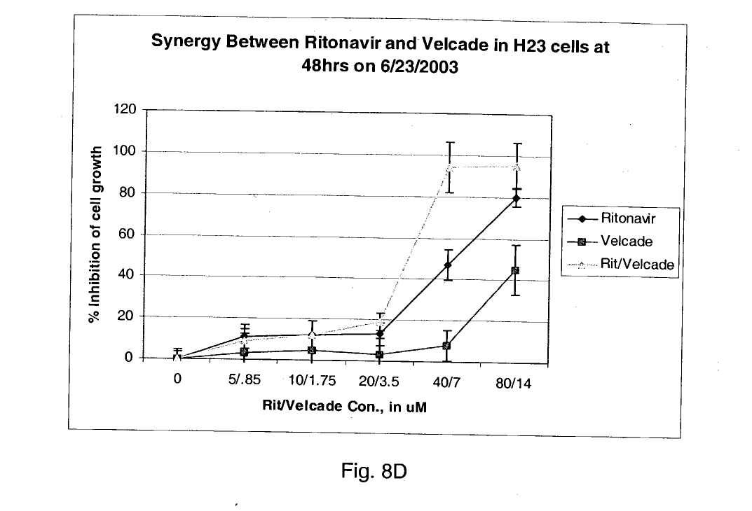

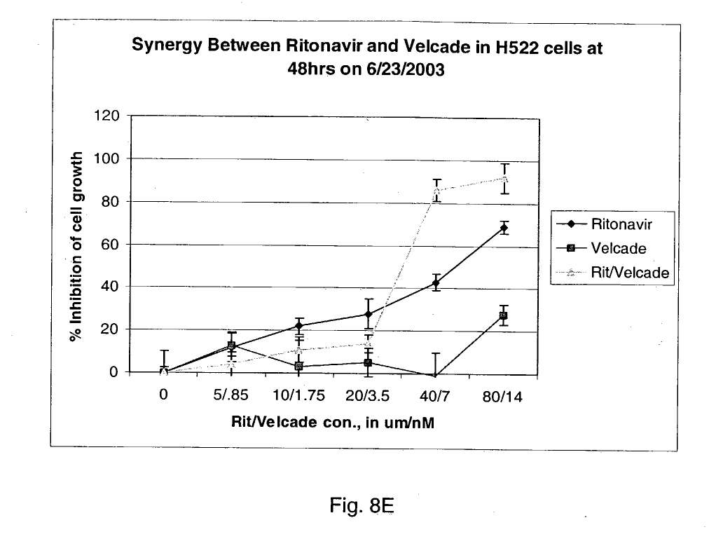

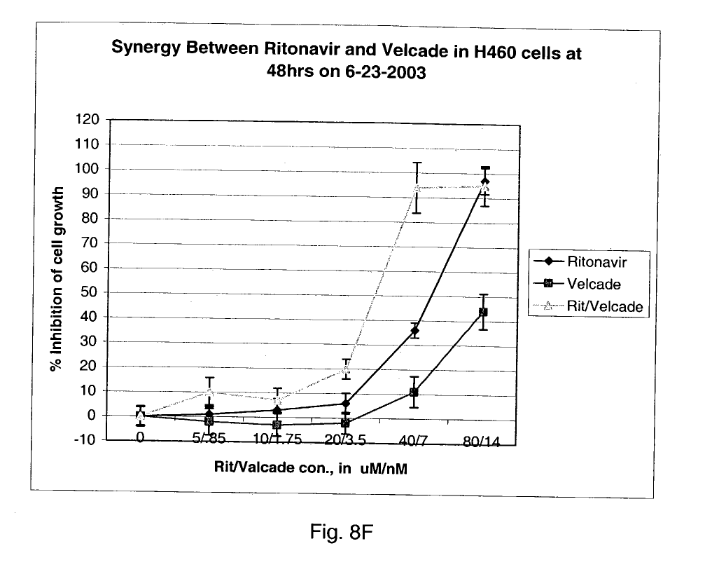

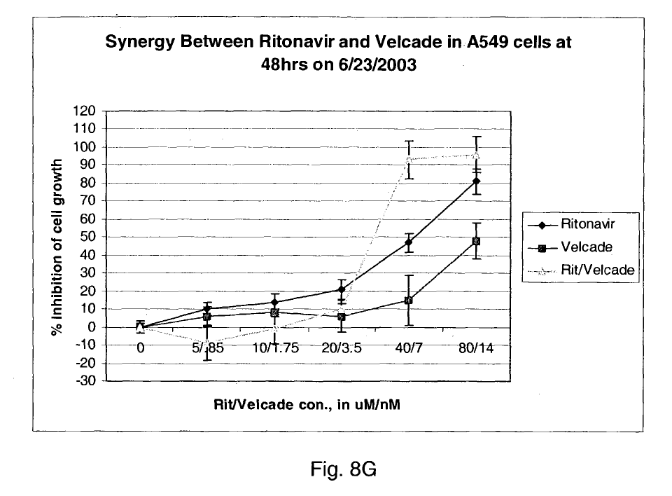

Ritonaivr was administered together with VELCADE™ in a variety of cell lines (Caco 2, which are colon cancer cells; and H23, H522, H460, and A549, which are non-small cell lung cancer cells). The results are depicted in FIGS. 8A-8G FIGS. 8A and 8B depict the IC₅₀s of Ritonavir alone and VELCADE™ alone, respectively. As illustrated in the figures, the co-administration of the two compounds produced a synergistic effect, demonstrating that simultaneously blocking both of the major protease systems in cancer cells can result in a substantial increase in log cell kill. (The synergy of ritonavir and VELCADE™ for each of Caco 2, H23, H522, H460, and A549 cells is depicted in FIGS. 8C-8G respectively. IC₅₀s are depicted in percent cell growth inhibition.)

### Example 15

### M-Calpain is Up-Regulated and Activated in Breast Cancer

Rabbit antibody reagents were developed and purified for immunohistochemical (IHC) studies. Ten of ten ductal breast cancer specimens tested were 1+ to 3+ positive for cytoplasmic m-calpain, while the cytoplasm of normal appearing adjacent mammary epithelium was mostly clear, with no basolateral staining.

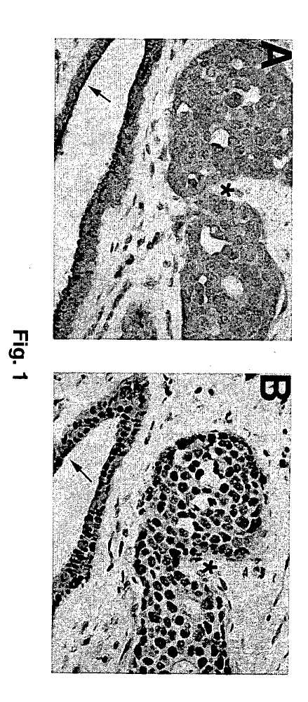

IHC data from five of these specimens are shown in the Table below and IHC analysis of one tumor is shown in FIG. 1, which depicts that m-calpain is diffusely cytoplasmic and activated in ductal breast cancer. FIG. 1A shows unautolyzed m-calpain detected by IHC of a ductal breast cancer using the affinity purified UMC antibody. And, FIG. 1B shows α-fodrin calpain cleavage product detected with the affinity purified PP1 antibody in an adjacent section to the section depicted in FIG. 1A. Arrows indicate the apical domain of normal-appearing breast epithelium. The tumor is marked with an asterisk.

Use of the Nuclear Fodrin Fragment Score for Calpain Activity: An antiserum, PP1, was made that specifically detects the 150 kDa upstream calpain-specific cleavage fragment or breakdown product (BDP) of human α-fodrin on 1 and 2D gels. PP1 does not detect not intact α-fodrin, but rather α-fodrin BDP antiserum detects intracellular calpain activity (Saido et al., J. Biol. Chem. 268:25239-25243, 1993). The fodrin fragment antiserum stained over 90% of the nuclei in tumors, but only 50% of the nuclei of adjacent ductal and terminal ductule epithelium. This finding is consistent with the determination that the nuclear fodrin fragment (NFF) score is 92% in colon cancers and 74% in adjacent normal appearing colonic epithelium (P<0.0009, n=14).

A similar increase in NFF from 50% in adjacent ducts to >90% in tumors was observed in preliminary analysis of mammary carcinomas arising in Cox-2 transgenic mammary tumors (Liu et al., J. Biol. Chem. 276:18563-18569, 2001)). This suggests that the Cox-2 carcinogenesis process can activate calpain. The co-localization of Cox-2 and m-calpain was tested in breast tumor tissue in adjacent sections. All of the tumors were at least 1+ positive for cytoplasmic Cox-2, while there was little or no staining of normal ductal epithelium. Cytoplasmic m-calpain and Cox-2 staining correlated in adjacent sections, suggesting that both proteins are likely to be present and increased in the majority of ductal breast cancers.

### Example 16

### Correlation Between M-Calpain and EGFR/her2 Up-Regulation in Breast Cancer

A summary of the IHC data demonstrating that m-calpain up-regulation and activation in breast cancer correlates with EGFR and her2 up-regulation is shown in the table below. The data is scored on a 0 to 3+scale. Four of 5 biopsies demonstrated either increased EGFR (2 of 5) or increased her2 (2 of 5), but not both. These data suggest that increased m-calpain correlates with increased EGFR or her2. Co-elevation of EGFR and her2, which occurs in about 10% of breast cancers (Suo et al., J. Pathol. 196:17-25, 2002), was not observed, likely due to the small sample size.

TABLE
Immunohistochemistry of m-calpain, calpain activity, EGFR,
ErbB2 (her2) and Cox-2 in ductal breast cancer cases*

| | | α-fodrin BDP | | | |
|---|---|---|---|---|---|
| Ductal breast | m-calpain | (calpain | EGF | ErbB2 | |
| cancer case | (antigen) | activity) | Receptor | (her2) | Cox-2 |
| #1 | 3+ | 2+ | 0 | 3+ | 1+ |
| #2 | 3+ | 2+ | 0 | 3+ | 1+ |
| #3 | 3+ | 2+ | 0 | 1+ | 2+ |
| #4 | 2+ | 2+ | 2+ | 1+ | 1+ |
| #5 | 3+ | 3+ | 3+ | 1+ | 2+ |

*Scale: 0 = no stain,
1+ >10% of cells stain weakly,
2+ all of cells stain moderately,
3+ all of cells stain intensely

Ritonavir is a calpain inhibitor: Ritonavir has demonstrated a 30 μM IC₅₀ (50% inhibition of proliferation at 48 h) for the proliferation of Caco 2 colon cancer cells. Furthermore, Caco 2 cells engineered to have reduced calpain activity demonstrate increased sensitivity to ritonavir (See the Table below, 2-3 and 0.5-11). The Caco 2 line exhibiting a calpain activity reduction of 80% has a ritonavir IC₅₀ of 15 μM (Table II). The sensitivity of Caco 2 lines to ritonavir correlates inversely with m-calpain activity (Table II), as would be expected if ritonavir is inhibiting calpain function in cells.

TABLE
Calpastatin over-expressing Caco 2 lines down-regulate surface EGFR, ErbB2 (her2),
ErbB3, phospho-ERK and phospho-AKT and are more sensitive to the calpain inhibitor,
ritonavir*

| | Calpain | | | | Phospho- | Phospho- | Ritonavir |
|---|---|---|---|---|---|---|---|
| Cell line | Activity | EGFR | ErbB2 (her2) | ErbB3 | ERK | AKT | IC₅₀ [μM] |
| C9 | 1 | 1 | 1 | 1 | 1 | 1 | 30 |
| control | | | | | | | |
| 2-3 | 0.73 ± 0.01 | 0.44 ± 0.060 | 0.68 ± 0.043 | 0.40 ± 0.086 | 0.58 ± 0.012 | 0.73 ± 0.023 | 20 |
| 0.5-11 | 0.22 ± 0.15 | 0.27 ± 0.12 | 0.49 ± 0.14 | 0.25 ± 0.30 | 0.59 ± 0.083 | 0.58 ± 0.043 | 15 |
| *The 2-3 and 0.5-11 lines are stably transfected with a calpastatin expression construct and the C9 with the pRC/CMV vector. Calpain activity was measured in situ in intact cells by a fluorometric assay (Potter et al., J. Cell Biol. 141: 647-662 (1998)). The EGFR, ErbB2 (her2), phospho-ERK and phospho-AKT were measured by western blotting. Ritonavir IC₅₀ was measured by MTT assay (Ohno et al., J. Immunol. Methods 145: 199-203 (1991)) in a | | | | | | | |
| # 96 well plate format (Monks et al., J. Natl. Cancer. Inst. 83: 757-766 (1991)). The IC₅₀ was defined as the concentration of ritonavir resulting in a 50% reduction in live cell number after 48 h of drug exposure. Ritonavir was purified from commercial sources. The molecular mass of the purified ritonavir was confirmed by mass spectroscopy | | | | | | | |

Ritonavir was tested in Caco 2 colon cells to determine whether Ritonavir blocks the cleavage of the ZGGL-AMC substrate. The results of this experiment are depicted in the Table below, showing that ritonavir does not block cleavage of the ZGGL-AMC substrate, whereas lactacystin (5 μM) blocks over 95% of substrate cleavage. In contrast, a calpain-specific inhibitor, PD150606, which interacts with the EF hand domain of m-calpain, blocks less than 30% of ZGGL-AMC cleavage. A control for cathepsins, the lysosomotropic agent NH₄Cl, has no effect on ZGGL-AMC cleavage, whereas ritonavir causes an increase, rather than a decrease in ZGGL-AMC cleavage.

TABLE III
Fluorometric assay of ZGGL-AMC cleavage by Caco 2 cells in the presence of
inhibitors of the proteasome, cathepsins or calpain*

| DMSO | Ritonavir | Ritonavir | Ritonavir | Ritonavir | Lactacystin | NH₄Cl | PD150606 |
|---|---|---|---|---|---|---|---|
| Control | 6 μM | 12 μM | 24 μM | 60 μM | 5 μM | 1 mM | 50 μM |
| 0.287 | 0.432 | 0.412 | 0.337 | 0.409 | 0.022 | 0.276 | 0.187 |

Units are pmol · s⁻¹ per 10⁶ cells.
The inhibitors, with the exception of NH₄Cl were added in DMSO solvent.

Gaedicke et al. (Cancer Res. 62:6901-6908, 2002) noted that although ritonavir inhibited purified 20S proteasomes, it increased the activity of the 26S proteasome, which has a regulatory complex and may more closely resemble the intracellular proteasome population. To determine whether the 20S proteasome preparations are contaminated with m-calpain, which can be constitutively active due to ERK phosphorylation (Glading et al., J. Biol. Chem. 276:23341-23348, 2001), purified bovine testicular 26S proteasome complexes were prepared using sequential glycerol gradient purification.

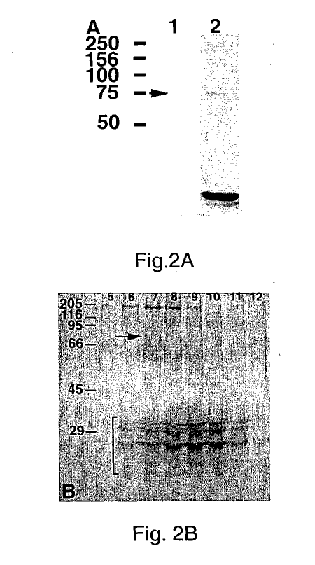

FIG. 2 shows silver stain of fractions of glycerol gradient purified bovine testicular proteasomes (Ustrell et al., Embo J 21:3516-3525, 2002)) repurified by repeat equilibrium centrifugation on a 10-40% glycerol gradient for 25 h at 25,000 rpm. The line indicates the mobility of the proteasome subunits and the arrow indicates a protein of 80 kDa Mᵣ. The peak fraction, #8, exhibiting the highest concentration of proteasome proteins, was assayed in FIG. 4A, blots 1 and 2.

Referring to FIGS. 2A and 2B, autolyzed m-calpain co-migrates with the 26S bovine proteasome on a glycerol gradient. FIG. 2A. shows glycerol gradient fractions of bovine testicular 26S proteasome assayed by western blotting with antisera to m-calpain. Blot 1 is a Western blot of peak 26S proteasome fraction probed with antibody UMC raised to amino acid residues 2-9 of unautolyzed human m-calpain. Blot 2 is a western blot of peak 26S proteasome fraction probed with antiserum raised to amino acid residues 2-21 of mammalian m-calpain (Croall et al., Biochim. Biophys. Acta. 1121:47-53, 1992). The autolysis site is between amino acids ala9 and lys10 (Dutt et al., FEBS Lett. 436:367-371, 1998) and so this antibody detects autolyzed m-calpain. The arrow indicates the 80 kDa positive control of porcine kidney m-calpain (Calbiochem) that reacted with both antisera. Molecular weight markers are listed (kDa). The 20 kDa breakdown product of m-calpain is also, seen in the porcine m-calpain preparation.

These findings suggest that activated m-calpain and the proteasome may exist in a complex. Such a complex may facilitate “substrate channeling” between the two proteases and could explain dual protease sensitivity of certain substrates, such as IκB-α (Han et al., J. Biol. Chem. 274:787-794, 1999; Pianetti et al., Oncogene 20:1287-1299, 2001).

### Example 17

Ritonavir Blocks the Proliferation of Breast Cancer Cells at Clinically Relevant Concentrations. The MCF7, T47D, MDA-231, SKBR-3 and MDA-436 lines were tested because m-calpain is lower in the first 2 lines and higher in the latter 3 lines (Table IV). The ritonavir IC₅₀ is 2-5-fold lower for the MCF7 and T47D lines, compared to MDA-231, SKBR-3, and MDA-436 (Table IV). This finding is consistent with the hypothesis that high m-calpain predicts ritonavir resistance. Confirmatory clonogenic assays measuring the LD₅₀ values for the cell lines in the Table below demonstrate LD₅₀ values 20-30 μM higher than the IC₅₀ values. Propidium iodide flow cytometry indicates that ritonavir induces cell cycle arrest in breast cancer lines at the IC₅₀ concentration of ritonavir. Annexin V/propidium iodide flow cytometry indicates that breast cancer lines undergo increased apoptosis at the ritonavir IC₅₀ and IC₈₀. Surface EGFR is down-regulated by ritonavir treatment of lines MDA-231, SKBR-3 and MDA-436 (Table below). Surface her2 is also down-regulated by ritonavir in the high her2 line (SKBR-3).

TABLE
Expression of m-calpain, EGRF and ErbB2 in Breast Cancer lines

| | | | | | Surface | Surface |
|---|---|---|---|---|---|---|
| | | | | ErbB2 | EGFR | her2 |
| Cell Line | ER Status | m-calpain | EGRF | (her2) | (Ritonavir) | Ritonavir |
| MCF7 | + | 1 | 1 | − | 1 | ND |
| T47D | + | 2 | 2.9 | − | 1 | ND |
| MDA-231 | − | 3.7 | 9.8 | − | 0.5 | ND |
| SKBR-3 | − | 7.8 | 2.1 | + | 0.36 | 0.4 |
| MDA-436 | − | 2.3 | 4.0 | + | 0.5 | ND |
| MDA-436LXSN | − | 2.3 | 5 | + | ND | ND |
| MDA-436IκBSR | − | 2.6 | 5.5 | + | ND | ND |

Ritonavir Resistance May Involve NF-κB Signaling: Chemotherapy resistance of human breast cancer cell lines has been linked to constitutive nuclear activity of NF-κB (Patel et al., Oncogene 19:4159-4169, 2000), which promotes expression of anti-apoptotic proteins including c-IAP2 and Mn-SOD. To determine whether constitutive nuclear NF-κB would confer resistance to ritonavir, the super-repressor of NF-κB (IκBSR) was over-expressed in MDA-436 breast cancer cells, which have constitutive nuclear NF-κB (“the 436-IκBSR line”). The 436-IκBSR line demonstrated a ritonavir IC₅₀ of 26 μM, compared to 42 μM for the vector control line 436-LXSN. The MDA-436 parental line demonstrated a ritonavir IC₅₀ of 40 μM (Table below). These data suggest that NF-κB may mediate resistance to ritonavir. Nonetheless, the over-expressed IκBSR does not completely block constitutive nuclear NF-κB. It is possible that the proteasome-specific inhibitor VELCADE™ (bortezomib) may yield a more complete block of nuclear NF-κB and enhance the cytolytic effects of ritonavir.

TABLE
Ritonavir sensitivity of breast cancer lines varies increases
with NF-κB inhibition

| | Constitutive | Ritonavir |
|---|---|---|
| Cell line | Nuclear NF-κB | IC₅₀ [μM] |
| MCF7 | + | 25 |
| T47D | + | 12 |
| MDA-231 | +++ | 50 |
| SKBR-3 | ++ | 60 |
| MDA-436 | ++++ | 40 |
| MDA-436LXSN | ++++ | 42 |
| MDA-436IκBSR | + | 26 |

A number of embodiments of the invention have been described. Nevertheless, it will be understood that various modifications may be made without departing from the spirit and scope of the invention. Accordingly, other embodiments are within the scope of the following claims.

What is claimed is:

1. A method for treating an HIV-negative patient who has cancer, the method comprising administering to the patient a therapeutically effective amount of a composition comprising
(a) a compound of Formula I 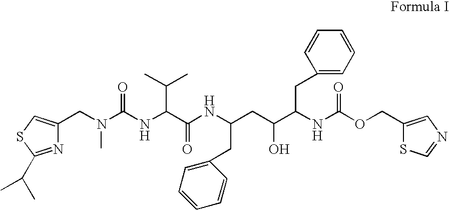
(b) ai compound of Formula II 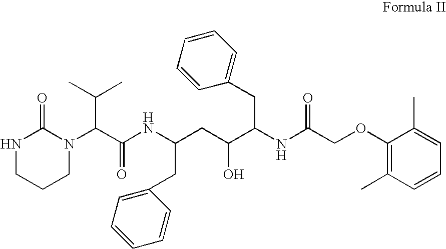
(c) a compound of Formula III 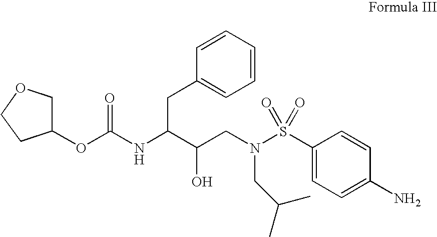
(d) a compound of Formula IV 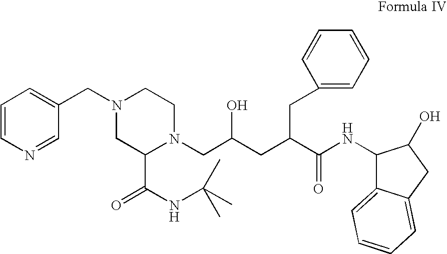
(e) a compound of formula V 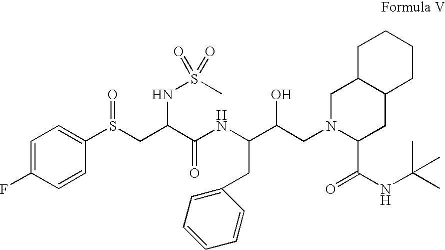
(f) a pharmaceutically acceptable salt of a compound of Formula I, Formula II, Formula III, Formula IV, or Formula V;
(g) a prodrug of a compound of Formula I, Formula II, Formula III, Formula IV, or Formula V.

2. The method of claim 1, wherein the composition comprises a compound of Formula I, a pharmaceutically acceptable salt of a compound of Formula I, or a prodrug of a compound of Formula I.

3. The method of claim 2, wherein the composition excludes a compound of Formula II, excludes a compound of Formula III, excludes a compound of Formula IV, excludes a compound of formula V, and excludes pharmaceutically acceptable salts or prodrugs of Formula II, Formula III, Formula IV, or Formula V.

4. The method of claim 1, wherein the composition comprises a compound of Formula I or a pharmaceutically acceptable salt or prodrug thereof and a compound of Formula II or a pharmaceutically acceptable salt or prodrug thereof.

5. The method of claim 4, wherein the amount, by weight, of the compound of Formula II or a pharmaceutically acceptable salt or prodrug thereof is approximately four times greater than the amount, by weight, of the compound of Formula I or a pharmaceutically acceptable salt or prodrug thereof.

6. The method of claim 1, wherein the composition comprises a compound of Formula III, a pharmaceutically acceptable salt of a compound of Formula III, or a prodrug of a compound of Formula III.

7. The method of claim 6, wherein the composition excludes a compound of Formula I, or a pharmaceutically acceptable salt or prodrug thereof, excludes a compound of Formula II, or a pharmaceutically acceptable salt or prodrug thereof, excludes a compound of Formula IV, or a pharmaceutically acceptable salt or prodrug thereof, and excludes a compound of Formula V, or a pharmaceutically acceptable salt or prodrug thereof.

8. The method of claim 1, wherein the composition comprises a compound of Formula IV, a pharmaceutically acceptable salt of a compound of Formula IV or a prodrug of a compound of Formula IV.

9. The method of claim 8, wherein the composition excludes a compound of Formula I, or a pharmaceutically acceptable salt or prodrug thereof, excludes a compound of Formula II, or a pharmaceutically acceptable salt or prodrug thereof, excludes a compound of Formula III, or a pharmaceutically acceptable salt or prodrug thereof, and excludes a compound of Formula V, or a pharmaceutically acceptable salt or prodrug thereof.

10. The method of claim 1, wherein the composition comprises a compound of Formula V, a pharmaceutically acceptable salt of a compound of Formula V or a prodrug of a compound of Formula V.

11. The method of claim 10, wherein the composition excludes a compound of Formula I, or a pharmaceutically acceptable salt or prodrug thereof, excludes a compound of Formula II, or a pharmaceutically acceptable salt or prodrug thereof, excludes a compound of Formula III, or a pharmaceutically acceptable salt or prodrug thereof, and excludes a compound of Formula IV, or a pharmaceutically acceptable salt or prodrug thereof.

12. The method of claim 1, wherein the composition further comprises a carrier, excipient, or diluent.

13. The method of claim 12, wherein the carrier, excipient, or diluent is a physiologically acceptable saline solution.

14. The method of claim 1, wherein the composition further comprises a pain relief agent, an antinausea agent, or an anticancer agent other than ritonavir, lopinavir, amprenavir, indinavir or saquinavir.

15. The method of claim 14, wherein the pain relief agent is a nonsteroidal anti-inflammatory drug (NSAID).

16. The method of claim 14, wherein the anticancer agent is paclitaxel, docetaxel, doxorubicin, liposomal doxorubicin, daunorubicin, epirubicin, fluorouracil, melphalan, cisplatin, carboplatin, cyclophosphamide, mitomycin-c, methotrexate, mitoxantrone, vinblastine, vincristine, vinorelbine, doxycycline, ifosfamide, teniposide, etoposide, bleomycin, leucovorin, cytarabine, dactinomycin, interferon alpha, streptozocin, prednisolone, interleukin-2 (IL-2), fludarabine, rituximab, campath (anti-CD 52), 2-CDA, anastrozole (Arinidex), tamoxifen, fulvestrant, reloxifine, letrozole, toremifene, flutamide, leuprolide, or procarbazine.

17. The method of claim 16, wherein the anticancer agent is paclitaxel or docetaxel.

18. The method of claim 16, wherein the anticancer agent is cisplatin or irinotecan.

19. The method of claim 1, wherein the composition further comprises an inhibitor of P-glycoprotein.

20. The method of claim 19, wherein the inhibitor of P-glycoprotein is Ketoconazole.

21. The method of claim 1, wherein the composition further comprises an inhibitor of an EGF receptor or of erbB2.

22. The method of claim 21, wherein the inhibitor is an antibody that specifically binds and antagonizes an EGF receptor or erbB2.

23. The method of claim 22, wherein the antibody is a humanized antibody.

24. The method of claim 21, wherein the inhibitor is antibody C225, antibody ADX-EGF, Iressa ZD1839, Tarceva OSI774, C1-1033, GW 572016, or EKB569.

25. The method of claim 1, wherein the composition further comprises a proteasome inhibitor.

26. The method of claim 25, wherein the proteasome inhibitor is bortezomib.

27. The method of claim 1, wherein the patient has a pancreatic cancer, a lung cancer, a breast cancer, a head and neck cancer, a prostate cancer, a colon cancer, a stomach cancer, an ovarian cancer, or a brain cancer.

28. The method of claim 1, wherein the patient has a cancer associated with resistance to known anticancer drug regimes.

29. The method of claim 28, wherein the cancer comprises cells that express a P-glycoprotein (MDR), a multidrug resistance-associated protein (MRP), or a breast cancer resistance protein (BCRP).

30. A method of predicting whether a patient who has cancer will respond to treatment with a composition comprising a calpain inhibitor, the method comprising:
(a) providing cells from a cancer cell line that is a model of the type of cancer the patient has and
(b) determining
(i) the level of expression or activity of m-calpain or
(ii) the level of expression or activity of an EGF receptor
in cells of the cell line, wherein a level m-calpain or of an EGF receptor that is higher than the level of expression or activity of m-calpain or an EGF receptor, respectively, in a reference cell, or population of reference cells, indicates that the patient is less likely to respond positively if treated with a composition comprising a calpain inhibitor.

31. A method of predicting whether a patient who has cancer will respond to treatment with a composition comprising a calpain inhibitor, the method comprising:
(a) providing cancerous cells from the patient and
(b) determining
(i) the level of expression or activity of m-calpain or
(ii) the level of expression or activity of an EGF receptor
in the cancerous cells from the patient, wherein a level that is higher than the level of expression or activity of m-calpain or an EGF receptor, respectively, in a reference cell, or population of reference cells, indicates that the patient is less likely to respond positively if treated with a composition comprising a calpain inhibitor.

32. A method of selecting a treatment regime for a patient who has cancer, the method comprising:
(a) providing cells from a cancer cell line that is a model of the type of cancer the patient has or providing cancerous cells from the patient
(b) exposing the cells from the cell line or the cancerous cells from the patient to at least two different compositions comprising a calpain inhibitor and
(c) determining which, if any, of the at least two different compositions is most effective in killing the cells from the cell line or the cancerous cells from the patient, reducing the motility of those cells, or reducing the rate at which they grow or proliferate.

33. The method of claim 32, wherein the compositions are identical except in the concentrations of the components they contain.

34. The method of claim 32, wherein step (b) is carried out in vivo.

35. The method of claim 32, wherein step (b) is carried out in cell culture.

36. The method of claim 30, wherein the composition comprises
(a) a compound of Formula I 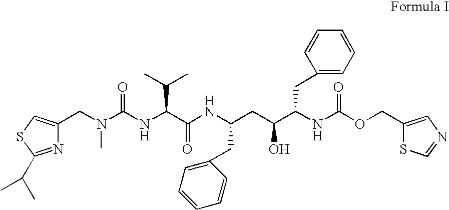
(b) a compound of Formula II 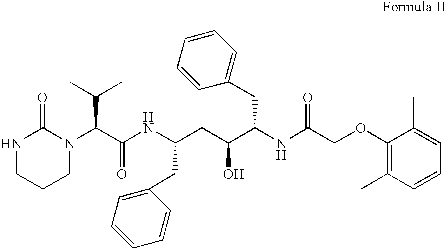
(c) a compound of Formula III 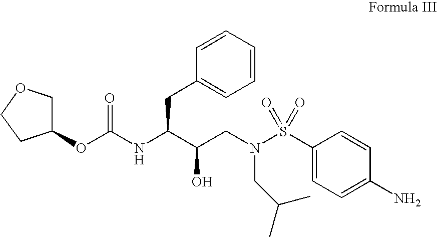
(d) a compound of Formula IV 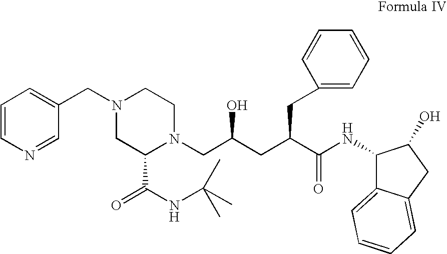
(e) a compound of formula V 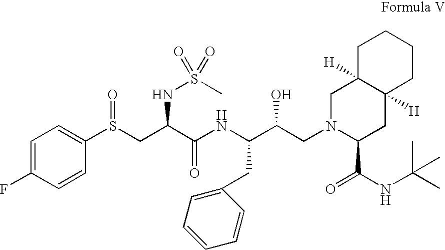
(f) a pharmaceutically acceptable salt of a compound of Formula I, Formula II, Formula III, Formula IV, or Formula V;
(g) a prodrug of a compound of Formula I, Formula II, Formula III, Formula IV, or Formula V.

37. The method of claim 36, wherein the composition comprises a compound of Formula I, a pharmaceutically acceptable salt of a compound of Formula I, or a prodrug of a compound of Formula I.

38. The method of claim 37, wherein the composition excludes a compound of Formula II, excludes a compound of Formula III, excludes a compound of Formula IV, excludes a compound of Formula V, and excludes pharmaceutically acceptable salts or prodrugs of Formula II, Formula III, Formula IV, or Formula V.

39. The method of claim 36, wherein the composition comprises a compound of Formula I or a pharmaceutically acceptable salt or prodrug thereof and a compound of Formula II or a pharmaceutically acceptable salt or prodrug thereof.

40. The method of claim 39, wherein the amount, by weight, of the compound of Formula II or a pharmaceutically acceptable salt or prodrug thereof is approximately four times greater than the amount, by weight, of the compound of Formula I or a pharmaceutically acceptable salt or prodrug thereof.

41. The method of claim 36, wherein the composition comprises a compound of Formula III, a pharmaceutically acceptable salt of a compound of Formula III, or a prodrug of a compound of Formula III.

42. The method of claim 41, wherein the composition excludes a compound of Formula I, or a pharmaceutically acceptable salt or prodrug thereof, excludes a compound of Formula II, or a pharmaceutically acceptable salt or prodrug thereof, excludes a compound of Formula IV, or a pharmaceutically acceptable salt or prodrug thereof, and excludes a compound of Formula V, or a pharmaceutically acceptable salt or prodrug thereof.

43. The method of claim 36, wherein the composition comprises a compound of Formula IV, a pharmaceutically acceptable salt of a compound of Formula IV or a prodrug of a compound of Formula IV.

44. The method of claim 43, wherein the composition excludes a compound of Formula I, or a pharmaceutically acceptable salt or prodrug thereof, excludes a compound of Formula II, or a pharmaceutically acceptable salt or prodrug thereof, excludes a compound of Formula III, or a pharmaceutically acceptable salt or prodrug thereof, and excludes a compound of Formula V, or a pharmaceutically acceptable salt or prodrug thereof.

45. The method of claim 36, wherein the composition comprises a compound of Formula V, a pharmaceutically acceptable salt of a compound of Formula IV or a prodrug of a compound of Formula V.

46. The method of claim 45, wherein the composition excludes a compound of Formula I, or a pharmaceutically acceptable salt or prodrug thereof, excludes a compound of Formula II, or a pharmaceutically acceptable salt or prodrug thereof, excludes a compound of Formula III, or a pharmaceutically acceptable salt or prodrug thereof, and excludes a compound of Formula IV, or a pharmaceutically acceptable salt or prodrug thereof.

47. The method of claim 36, wherein the composition further comprises a carrier, excipient, or diluent.

48. The method of claim 39, wherein the carrier, excipient, or diluent is a physiologically acceptable saline solution.

49. The method of claim 36, wherein the composition is tested in conjunction with a pain relief agent, an antinausea agent, an anticancer agent other than ritonavir, lopinavir, or amprenavir, an inhibitor of P-glycoprotein, an inhibitor of an EGF receptor, or a proteasome inhibitor.

50. The method of claim 30, wherein the cancer is a pancreatic cancer, a lung cancer, a breast cancer, a head and neck cancer, a prostate cancer, a colon cancer, a stomach cancer, an ovarian cancer, or a brain cancer.

51. The method of claim 30, wherein the cancer is associated with resistance to known anticancer drug regimes.

52. The method of claim 30, wherein the cancer comprises cells that express a P-glycoprotein (MDR), a multidrug resistance-associated protein (MRP), or a breast cancer resistance protein (BCRP).

53. The method of claim 30, wherein the level of m-calpain or the level of an EGF receptor is measured by assessing m-calpain or EGF receptor mRNA, respectively.

54. The method of claim 31, wherein the level of m-calpain or the level of an EGF receptor is measured by assessing m-calpain or EGF receptor mRNA, respectively.

55. The method of claim 30, wherein the level of m-calpain or the level of an EGF receptor is measured by assessing m-calpain or EGF receptor protein, respectively.

56 The method of claim 31, wherein the level of m-calpain or the level of an EGF receptor is measured by assessing m-calpain or EGF receptor protein, respectively.

57 A method of treating an HIV-negative patient who has cancer comprising administering to the patient a therapeutically effective amount of a composition comprising a compound of Formula I or a pharamaceutically acceptable salt or prodrug thereof, and a proteasome inhibitor.

58 The method of claim 57 wherein the proteasome inhibitor is bortezomib.

59 A method of treating an HIV-negative patient who has cancer comprising administering to the patient a therapeutically effective amount of a composition comprising a compound of Formula I or a pharamaceutically acceptable salt or prodrug thereof, and a Cox-2 inhibitor.

60 A method of treating an HIV-negative patient who has cancer comprising administering to the patient a therapeutically effective amount of a composition comprising a compound of Formula I or a pharamaceutically acceptable salt or prodrug thereof, and docetaxel.
